(19) United States
(12) Patent Application Publication
(10) Pub. No.: US 2025/0169676 A1
(43) Pub. Date: May 29, 2025

(54) MEDICAL SUPPORT DEVICE, ENDOSCOPE, MEDICAL SUPPORT METHOD, AND PROGRAM

(71) Applicant: FUJIFILM CORPORATION, Tokyo (JP)

(72) Inventor: Kentaro OSHIRO, Kanagawa (JP)

(21) Appl. No.: 19/040,863

(22) Filed: Jan. 30, 2025

Related U.S. Application Data
(63) Continuation of application No. PCT/JP2023/026214, filed on Jul. 18, 2023.

(30) Foreign Application Priority Data
Aug. 30, 2022 (JP) 2022-137263

(51) Int. Cl. A61B 1/00

(52) U.S. Cl. CPC A61B 1/000094; A61B 1/0005

(57) ABSTRACT

A medical support device includes a processor. The processor is configured to: recognize a plurality of parts in an observation target on the basis of a plurality of medical images including the observation target; and in a case where an unrecognized part in the observation target is present in the plurality of parts, output unrecognized information capable of specifying that the unrecognized part is present.

### CROSS-REFERENCE TO RELATED APPLICATIONS

This application is a continuation application of International Application No. PCT/JP2023/026214, filed Jul. 18, 2023, the disclosure of which is incorporated herein by reference in its entirety. Further, this application claims priority from Japanese Patent Application No. 2022-137263, filed Aug. 30, 2022, the disclosure of which is incorporated herein by reference in its entirety.

### BACKGROUND

### 1. Technical Field

The technology of the present disclosure relates to a medical support device, an endoscope, a medical support method, and a program.

### 2. Related Art

WO2021/176664A discloses an examination support system comprising an acquisition unit that acquires an image captured by an imaging unit of an endoscope in a luminal organ of a patient and spatial disposition information of a distal end of an insertion portion of the endoscope, an abundance ratio calculation unit that calculates an abundance ratio of a polyp in an unobserved region in the luminal organ which is specified on the basis of at least the image and the spatial disposition information, and an examination plan creation unit that creates an examination plan including a schedule for a next examination of the luminal organ on the basis of at least the abundance ratio of the polyp.

JP2015-198928A discloses a medical image processing device that displays at least one medical image obtained by imaging a subject and that comprises a position detection unit that detects a position of a characteristic local structure of a human body from the medical image, a check information determination unit that determines check information indicating a local structure to be checked, an interpretation determination unit that determines whether or not the local structure to be checked, which is indicated by the check information, has been interpreted on the basis of the position of the local structure detected from the medical image, and a display unit that displays a determination result of the interpretation determination unit.

JP2015-217120A discloses an image diagnosis support device comprising a display unit that displays a tomographic image obtained from a three-dimensional medical image on a display screen, a detection unit that detects a gaze position of a user on the display screen, a determination unit that determines an observed region in the tomographic image on the basis of the gaze position detected by the detection unit, and an identification unit that identifies the observed region in the three-dimensional medical image on the basis of the observed region in the tomographic image determined by the determination unit.

### SUMMARY

An embodiment according to the technology of the present disclosure provides a medical support device, an endoscope, a medical support method, and a program that can contribute to suppressing omission of recognition for a part in an observation target.

According to a first aspect of the technology of the present disclosure, there is provided a medical support device comprising a processor, in which the processor is configured to: recognize a plurality of parts in an observation target on the basis of a plurality of medical images including the observation target; and, in a case where an unrecognized part in the observation target is present in the plurality of parts, output unrecognized information capable of specifying that the unrecognized part is present.

According to a second aspect of the technology of the present disclosure, in the medical support device according to the first aspect, the plurality of parts include a subsequent part that is scheduled to be recognized by the processor after the unrecognized part, and the processor is configured to output the unrecognized information on condition that the subsequent part is recognized.

According to a third aspect of the technology of the present disclosure, in the medical support device according to the first aspect or the second aspect, the processor is configured to output the unrecognized information on the basis of a first order, which is an order in which the plurality of parts are recognized by the processor, and a second order, which is an order in which a plurality of scheduled parts that include the unrecognized part and that are scheduled to be recognized by the processor are recognized by the processor.

According to a fourth aspect of the technology of the present disclosure, in the medical support device according to any one of the first to third aspects, importance is assigned to the plurality of parts, and the unrecognized information includes importance information capable of specifying the importance.

According to a fifth aspect of the technology of the present disclosure, in the medical support device according to the fourth aspect, the importance is determined in response to an instruction given from an outside.

According to a sixth aspect of the technology of the present disclosure, in the medical support device according to the fourth aspect or the fifth aspect, the importance is determined according to data of a past examination performed on the plurality of parts.

According to a seventh aspect of the technology of the present disclosure, in the medical support device according to any one of the fourth to sixth aspects, the importance is determined according to a position of the unrecognized part in the observation target.

According to an eighth aspect of the technology of the present disclosure, in the medical support device according to any one of the fourth to seventh aspects, the importance corresponding to a part which is scheduled to be recognized by the processor before a designated part, among the plurality of parts, is higher than the importance corresponding to a part which is scheduled to be recognized after the designated part, among the plurality of parts.

According to a ninth aspect of the technology of the present disclosure, in the medical support device according to any one of the fourth to eighth aspects, the importance corresponding to a part which is determined to be a part for which omission of recognition is typically likely to occur, among the plurality of parts, is higher than the importance corresponding to a part which is determined to be a part for which omission of recognition is typically unlikely to occur, among the plurality of parts.

According to a tenth aspect of the technology of the present disclosure, in the medical support device according to any one of the fourth to ninth aspects, the plurality of parts are classified into a major category and a minor category included in the major category, and the importance corresponding to a part which is classified into the minor category, among the plurality of parts, is higher than the importance corresponding to a part which is classified into the major category, among the plurality of parts.

According to an eleventh aspect of the technology of the present disclosure, in the medical support device according to any one of the first to tenth aspects, the plurality of parts are classified into a major category and a minor category included in the major category, and the unrecognized part is a part which is classified into the minor category, among the plurality of parts.

According to a twelfth aspect of the technology of the present disclosure, in the medical support device according to the eleventh aspect, the major category is roughly classified into a first major category and a second major category, the part classified into the second major category is scheduled to be recognized by the processor after the part classified into the first major category, the unrecognized part is a part which belongs to the minor category included in the first major category, among the plurality of parts, and the processor is configured to output the unrecognized information on condition that a part which is classified into the second major category, among the plurality of parts, is recognized.

According to a thirteenth aspect of the technology of the present disclosure, in the medical support device according to the eleventh aspect or the twelfth aspect, the plurality of parts include a plurality of minor category parts classified into the minor category, the plurality of minor category parts include a first minor category part and a second minor category part that is scheduled to be recognized by the processor after the first minor category part, the unrecognized part is the first minor category part, and the processor is configured to output the unrecognized information on condition that the second minor category part is recognized.

According to a fourteenth aspect of the technology of the present disclosure, in the medical support device according to the eleventh aspect or the twelfth aspect, the plurality of parts include a plurality of minor category parts belonging to the minor category, the plurality of minor category parts include a first minor category part and a plurality of second minor category parts that are scheduled to be recognized by the processor after the first minor category part, the unrecognized part is the first minor category part, and the processor is configured to output the unrecognized information on condition that the plurality of second minor category parts are recognized.

According to a fifteenth aspect of the technology of the present disclosure, in the medical support device according to any one of the first to fourteenth aspects, an output destination of the unrecognized information includes a display device.

According to a sixteenth aspect of the technology of the present disclosure, in the medical support device according to the fifteenth aspect, the unrecognized information includes a first image capable of specifying the unrecognized part and a second image capable of specifying parts other than the unrecognized part, among the plurality of parts, and the first image and the second image are displayed on the display device in an aspect in which the first image and the second image are distinguishable.

According to a seventeenth aspect of the technology of the present disclosure, in the medical support device according to the sixteenth aspect, a schematic view in which the observation target is divided into a plurality of regions corresponding to the plurality of parts is displayed on the display device, and the first image and the second image are displayed in the schematic view in an aspect in which the first image and the second image are distinguishable.

According to an eighteenth aspect of the technology of the present disclosure, in the medical support device according to the seventeenth aspect, the observation target is a luminal organ, and the schematic view is a first schematic view showing a schematic aspect of at least one route for observing the luminal organ, a second schematic view perspectively showing a schematic aspect of the luminal organ, and/or a third schematic view showing an aspect in which the luminal organ is schematically developed.

According to a nineteenth aspect of the technology of the present disclosure, in the medical support device according to any one of the first to eighteenth aspects, the first image is displayed on the display device in a state in which the first image is emphasized more than the second image.

According to a twentieth aspect of the technology of the present disclosure, in the medical support device according to any one of the sixteenth to nineteenth aspects, importance is assigned to the plurality of parts, and a display aspect of the first image differs depending on the importance.

According to a twenty-first aspect of the technology of the present disclosure, in the medical support device according to any one of the sixteenth to twentieth aspects, a display aspect of the first image differs depending on a type of the unrecognized part.

According to a twenty-second aspect of the technology of the present disclosure, in the medical support device according to any one of the first to twenty-first aspects, the medical image is an image obtained from an endoscope inserted into a body, and the processor is configured to: in a case in which a first part on an upstream side in an insertion direction of the endoscope inserted into the body and a second part on a downstream side are sequentially recognized, output the unrecognized information along a first route determined from the upstream side to the downstream side in the insertion direction; and, in a case in which a third part on the downstream side in the insertion direction and a fourth part on the upstream side are sequentially recognized, output the unrecognized information along a second route determined from the downstream side to the upstream side in the insertion direction.

According to a twenty-third aspect of the technology of the present disclosure, there is provided an endoscope comprising the medical support device according to any one of the first to twenty-second aspects; and an image acquisition device that acquires an endoscopic image as the medical image.

According to a twenty-fourth aspect of the technology of the present disclosure, there is provided a medical support method comprising: recognizing a plurality of parts in an observation target on the basis of a plurality of medical images including the observation target; and, in a case where an unrecognized part in the observation target is present in the plurality of parts, outputting unrecognized information capable of specifying that the unrecognized part is present.

According to a twenty-fifth aspect of the technology of the present disclosure, there is provided a program causing a computer to execute a process comprising: recognizing a plurality of parts in an observation target on the basis of a plurality of medical images including the observation target; and, in a case where an unrecognized part in the observation target is present in the plurality of parts, outputting unrecognized information capable of specifying that the unrecognized part is present.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example of an overall configuration of the endoscope system;

FIG. 3 is a block diagram illustrating an example of a hardware configuration of an electrical system of the endoscope system;

FIG. 4 is a block diagram illustrating an example of functions of main units of a processor included in an endoscope;

FIG. 5 is a conceptual diagram illustrating an example of a correlation among a camera, an NVM, an image acquisition unit, and a recognition unit;

FIG. 6 is a conceptual diagram illustrating an example of a configuration of a recognition part check table;

FIG. 7 is a conceptual diagram illustrating an example of a configuration of an importance table;

FIG. 8 is a conceptual diagram illustrating an example of a correlation between a control unit and a display device;

FIG. 9 is a conceptual diagram illustrating an example of a medical support image displayed on a screen of the display device;

FIG. 10 is a flowchart illustrating an example of a flow of a medical support process;

FIG. 11 is a conceptual diagram illustrating a first modification example of the medical support image displayed on the screen of the display device; and

FIG. 12 is a conceptual diagram illustrating a second modification example of the medical support image displayed on the screen of the display device.

### DETAILED DESCRIPTION

Hereinafter, examples of embodiments of a medical support device, an endoscope, a medical support method, and a program according to the technology of the present disclosure will be described with reference to the accompanying drawings.

First, terms used in the following description will be described.

CPU is an abbreviation of “central processing unit”. GPU is an abbreviation of “graphics processing unit”. RAM is an abbreviation of “random-access memory”. NVM is an abbreviation of “non-volatile memory”. EEPROM is an abbreviation of “electrically erasable programmable read-only memory”. ASIC is an abbreviation of “application-specific integrated circuit”. PLD is an abbreviation of “programmable logic device”. FPGA is an abbreviation of “field-programmable gate array”. SoC is an abbreviation of “system-on-a-chip”. SSD is an abbreviation of “solid-state drive”. USB is an abbreviation of “Universal Serial Bus”. HDD is an abbreviation of “hard disk drive”. EL is an abbreviation of “electro-luminescence”. CMOS is an abbreviation of “complementary metal-oxide-semiconductor”. CCD is an abbreviation of “charge-coupled device”. AI is an abbreviation of “artificial intelligence”. BLI is an abbreviation of “blue light imaging”. LCI is an abbreviation of “linked color imaging”. I/F is an abbreviation of “interface”. FIFO is an abbreviation of “first in, first out”.

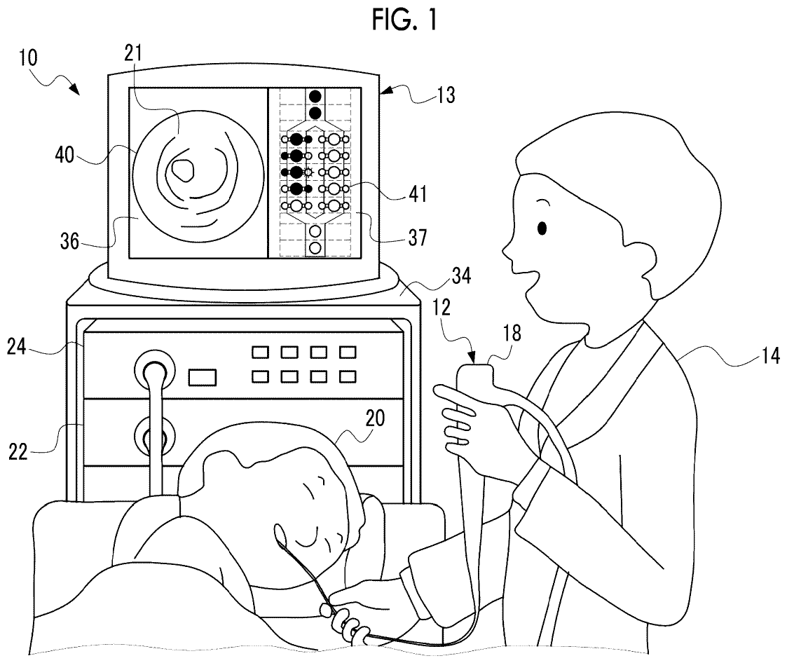

For example, as illustrated in FIG. 1, an endoscope system 10 comprises an endoscope 12 and a display device 13. The endoscope 12 is used by a doctor 14 in endoscopy. The endoscope 12 is connected to a communication device (not illustrated) such that it can communicate, and information obtained by the endoscope 12 is transmitted to the communication device. The communication device receives the information transmitted from the endoscope 12 and performs a process using the received information (for example, a process of recording the information in an electronic medical record or the like).

The endoscope 12 comprises an endoscope main body 18. The endoscope 12 is an apparatus for performing a medical treatment on an observation target 21 (for example, an upper digestive organ) included in a body of a subject 20 (for example, a patient) using the endoscope main body 18. The observation target 21 is an object observed by the doctor 14. The endoscope main body 18 is inserted into the body of the subject 20. The endoscope 12 directs the endoscope main body 18 inserted into the body of the subject 20 to image the observation target 21 in the body of the subject 20 and performs various medical treatments on the observation target 21 as necessary. The endoscope 12 is an example of an “endoscope” according to the technology of the present disclosure.

The endoscope 12 images the inside of the body of the subject 20 to acquire an image showing an aspect of the inside of the body and outputs the image. In the example illustrated in FIG. 1, an upper endoscope is given as an example of the endoscope 12. In addition, the upper endoscope is only an example, and the technology of the present disclosure can be established even in a case where the endoscope 12 is another type of endoscope such as a lower gastrointestinal endoscope or a bronchoscope.

Further, in the present embodiment, the endoscope 12 is an endoscope having an optical imaging function that irradiates the inside of the body with light and captures light reflected by the observation target 21. However, this is only an example, and the technology of the present disclosure is established even in a case where the endoscope 12 is an ultrasonic endoscope. In addition, the technology of the present disclosure is established even in a case where a modality that generates a frame for a medical examination or surgery (for example, a radiographic image obtained by performing imaging using radiation or the like or an ultrasound image based on reflected waves of ultrasonic waves emitted from the outside of the subject 20) is used instead of the endoscope 12. Further, the frame for a medical examination or surgery is an example of a “medical image” according to the technology of the present disclosure.

The endoscope 12 comprises a control device 22 and a light source device 24. The control device 22 and the light source device 24 are installed in a wagon 34. A plurality of tables are provided in the wagon 34 along a vertical direction, and the control device 22 and the light source device 24 are installed from a lower table to an upper table. In addition, the display device 13 is installed on the uppermost table in the wagon 34.

The display device 13 displays various types of information including images. An example of the display device 13 is a liquid-crystal display or an EL display. In addition, a tablet terminal with a display may be used instead of the display device 13 or together with the display device 13.

A plurality of screens are displayed side by side on the display device 13. In the example illustrated in FIG. 1, screens 36 and 37 are illustrated. An endoscopic image 40 obtained by the endoscope 12 is displayed on the screen 36. The observation target 21 is included in the endoscopic image 40. The endoscopic image 40 is an image generated by imaging the observation target 21 with the endoscope 12 in the body of the subject 20. An example of the observation target 21 is the upper digestive organ. Hereinafter, for convenience of explanation, a stomach will be described as an example of the upper digestive organ. The stomach is an example of a “luminal organ” according to the technology of the present disclosure. In addition, the stomach is only an example, and the observation target 21 may be any region that can be imaged by the endoscope 12. A luminal organ, such as a large intestine, a small intestine, a duodenum, an esophagus, or a bronchus, is given as an example of the region that can be imaged by the endoscope 12. The endoscopic image 40 is an example of the “medical image” according to the technology of the present disclosure.

A video including the endoscopic images 40 of a plurality of frames is displayed on the screen 36. That is, the endoscopic images 40 of a plurality of frames are displayed on the screen 36 at a predetermined frame rate (for example, several tens of frames/sec).

A medical support image 41 is displayed on the screen 37. The medical support image 41 is an image that is referred to by the doctor 14 during endoscopy. The medical support image 41 is referred to by the doctor 14 in order to check whether or not there is omission of observation for a plurality of parts that are scheduled to be observed during the endoscopy.

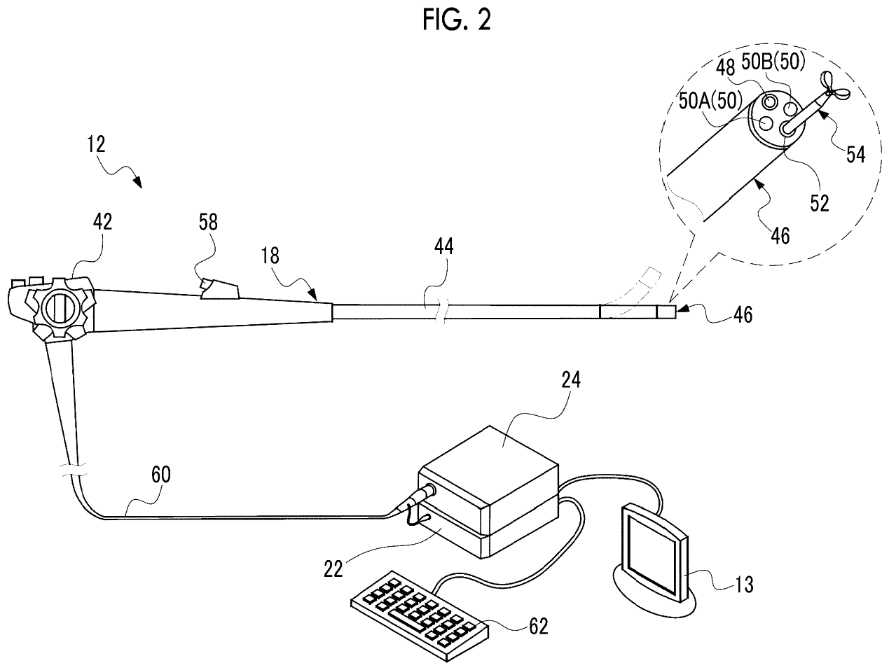

For example, as illustrated in FIG. 2, the endoscope 12 comprises an operation portion 42 and an insertion portion 44. The insertion portion 44 is partially curved by the operation of the operation portion 42. The insertion portion 44 is inserted while being curved according to the shape of the observation target 21 (for example, the shape of the stomach) in response to the operation of the operation portion 42 by the doctor 14.

A camera 48, an illumination device 50, and a treatment opening 52 are provided in a distal end part 46 of the insertion portion 44. The camera 48 is a device that images the inside of the body of the subject 20 to acquire the endoscopic image 40 as the medical image. The camera 48 is an example of an “image acquisition device” according to the technology of the present disclosure. An example of the camera 48 is a CMOS camera. However, this is only an example, and the camera 48 may be other types of cameras such as CCD cameras.

The illumination device 50 has illumination windows 50A and 50B. The illumination device 50 emits light through the illumination windows 50A and 50B. Examples of the type of light emitted from the illumination device 50 include visible light (for example, white light) and invisible light (for example, near-infrared light). In addition, the illumination device 50 emits special light through the illumination windows 50A and 50B. Examples of the special light include light for BLI and/or light for LCI. The camera 48 images the inside of the subject 20 using an optical method in a state in which the inside of the subject 20 is irradiated with light by the illumination device 50.

The treatment opening 52 is used as a treatment tool protruding port through which a treatment tool 54 protrudes from the distal end part 46, a suction port for sucking, for example, blood and body waste, and a delivery port for sending out a fluid.

The treatment tool 54 protrudes from the treatment opening 52 in response to the operation of the doctor 14. The treatment tool 54 is inserted into the insertion portion 44 through a treatment tool insertion opening 58. The treatment tool 54 passes through the insertion portion 44 through the treatment tool insertion opening 58 and protrudes from the treatment opening 52 into the body of the subject 20. In the example illustrated in FIG. 2, as the treatment tool 54, forceps protrude from the treatment opening 52. The forceps are only an example of the treatment tool 54, and other examples of the treatment tool 54 include a wire, a scalpel, and an ultrasound probe.

A suction pump (not illustrated) is connected to the endoscope main body 18, and blood, body waste, and the like of the observation target 21 are sucked by the suction force of the suction pump through the treatment opening 52. The suction force of the suction pump is controlled in response to an instruction given from the doctor 14 to the endoscope 12 through, for example, the operation portion 42.

A supply pump (not illustrated) is connected to the endoscope main body 18, and the fluid (for example, gas and/or liquid) is supplied into the endoscope main body 18 by the supply pump. The fluid supplied from the supply pump to the endoscope main body 18 is sent out through the treatment opening 52. Gas (for example, air) and liquid (for example, physiological saline) are selectively sent out as the fluid from the treatment opening 52 into the body in response to an instruction given from the doctor 14 to the endoscope 12 through the operation portion 42 or the like. The amount of the fluid sent out is controlled in response to an instruction given from the doctor 14 to the endoscope 12 through the operation portion 42 or the like.

In addition, here, an example of the form in which the treatment opening 52 is used as the treatment tool protruding port, the suction port, and the delivery port is given. However, this is only an example, and the treatment tool protruding port, the suction port, and the delivery port may be separately provided in the distal end part 46, or the treatment tool protruding port and an opening that serves as the suction port and the delivery port may be provided in the distal end part 46.

The endoscope main body 18 is connected to the control device 22 and the light source device 24 through a universal cord 60. The display device 13 and a receiving device 62 are connected to the control device 22. The receiving device 62 receives an instruction from the user and outputs the received instruction as an electric signal. In the example illustrated in FIG. 2, a keyboard is given as an example of the receiving device 62. However, this is only an example, and the receiving device 62 may be, for example, a mouse, a touch panel, a foot switch and/or a microphone.

The control device 22 controls the entire endoscope 12. For example, the control device 22 controls the light source device 24, transmits and receives various signals to and from the camera 48, or displays various types of information on the display device 13. The light source device 24 emits light under the control of the control device 22 and supplies the light to the illumination device 50. A light guide is provided in the illumination device 50, and the light supplied from the light source device 24 is emitted from the illumination windows 50A and 50B through the light guide. The control device 22 directs the camera 48 to perform imaging, acquires the endoscopic image 40 (see FIG. 1) from the camera 48, and outputs the endoscopic image 40 to a predetermined output destination (for example, the display device 13).

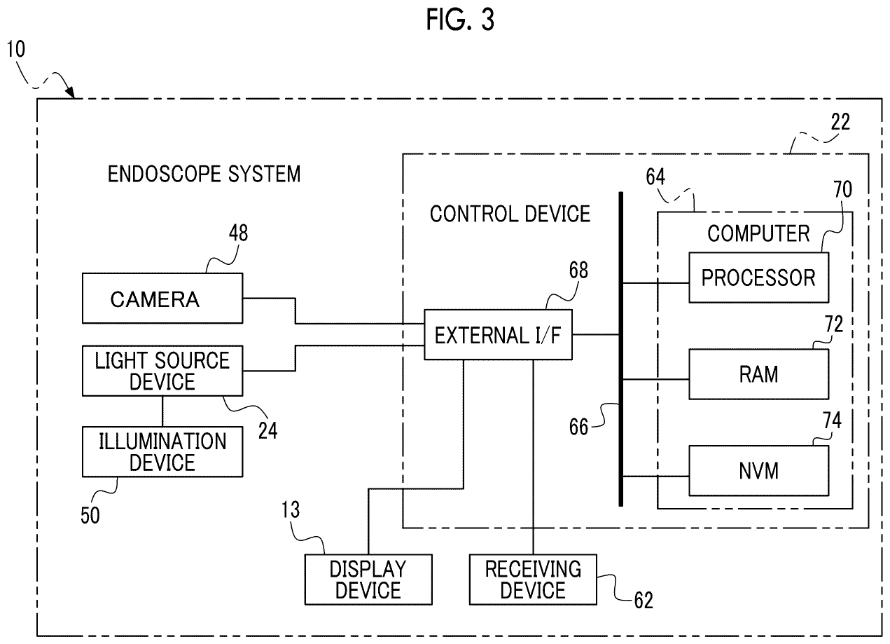

For example, as illustrated in FIG. 3, the control device 22 comprises a computer 64. The computer 64 is an example of a “medical support device” and a “computer” according to the technology of the present disclosure. The computer 64 comprises a processor 70, a RAM 72, and an NVM 74. The processor 70, the RAM 72, and the NVM 74 are electrically connected to each other. The processor 70 is an example of a “processor” according to the technology of the present disclosure.

The control device 22 comprises the computer 64, a bus 66, and an external I/F 68. The computer 64 comprises the processor 70, the RAM 72, and the NVM 74. The processor 70, the RAM 72, the NVM 74, and the external I/F 68 are connected to the bus 66.

For example, the processor 70 includes a CPU and a GPU and controls the entire control device 22. The GPU operates under the control of the CPU and is in charge of, for example, performing various processes of a graphic system and performing calculation using a neural network. In addition, the processor 70 may be one or more CPUs with which the functions of the GPU have been integrated or may be one or more CPUs with which the functions of the GPU have not been integrated.

The RAM 72 is a memory that temporarily stores information and is used as a work memory by the processor 70. The NVM 74 is a non-volatile storage device that stores, for example, various programs and various parameters. An example of the NVM 74 is a flash memory (for example, an EEPROM and/or an SSD). In addition, the flash memory is only an example, and other non-volatile storage devices, such as HDDs, or a combination of two or more types of non-volatile storage devices may be used.

The external I/F 68 transmits and receives various types of information between a device (hereinafter, also referred to as an “external device”) outside the control device 22 and the processor 70. An example of the external I/F 68 is a USB interface.

As one of the external devices, the camera 48 is connected to the external I/F 68, and the external I/F 68 transmits and receives various types of information between the camera 48 and the processor 70. The processor 70 controls the camera 48 via the external I/F 68. In addition, the processor 70 acquires the endoscopic image 40 (see FIG. 1) obtained by imaging the inside of the subject 20 with the camera 48 through the external I/F 68.

As one of the external devices, the light source device 24 is connected to the external I/F 68, and the external I/F 68 transmits and receives various types of information between the light source device 24 and the processor 70. The light source device 24 supplies light to the illumination device 50 under the control of the processor 70. The illumination device 50 performs irradiation with the light supplied from the light source device 24.

As one of the external devices, the display device 13 is connected to the external I/F 68, and the processor 70 controls the display device 13 through the external I/F 68 such that the display device 13 displays various types of information.

As one of the external devices, the receiving device 62 is connected to the external I/F 68. The processor 70 acquires the instruction received by the receiving device 62 through the external I/F 68 and performs a process corresponding to the acquired instruction.

However, in general, in endoscopy, a lesion is detected by using an image recognition process (for example, an AI-type image recognition process). In some cases, for example, a treatment for cutting out the lesion is performed. In addition, in endoscopy, since the doctor 14 performs the operation of the insertion portion 44 of the endoscope 12 and the differentiation of a lesion at the same time, the burden on the doctor 14 is large, and there is a concern that the lesion will be overlooked. In order to prevent the lesion from being overlooked, it is important that a plurality of parts scheduled in advance in the observation target 21 are recognized by the image recognition process without omission. However, it is very difficult for the doctor 14 to proceed with the work while checking whether or not a plurality of parts scheduled in advance have been recognized by the image recognition process without omission.

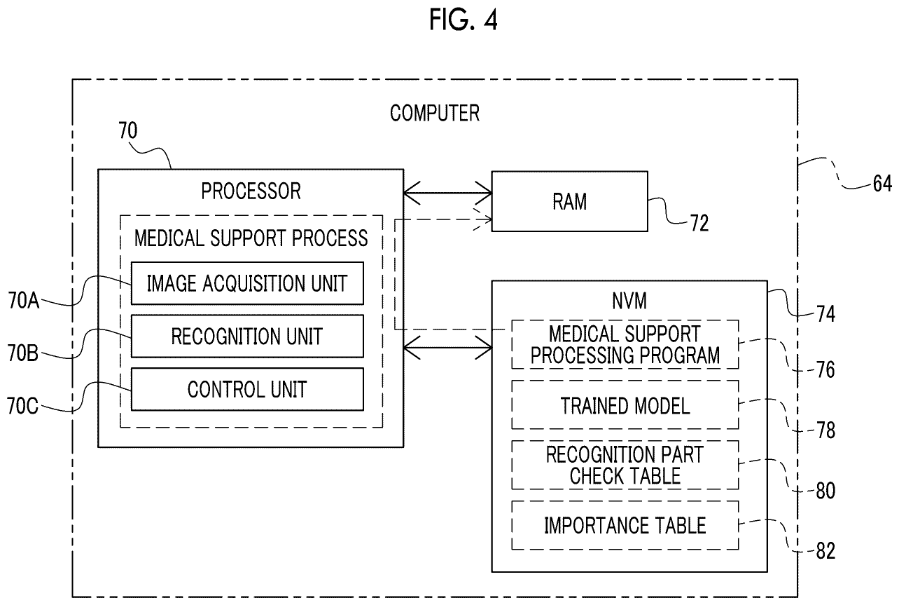

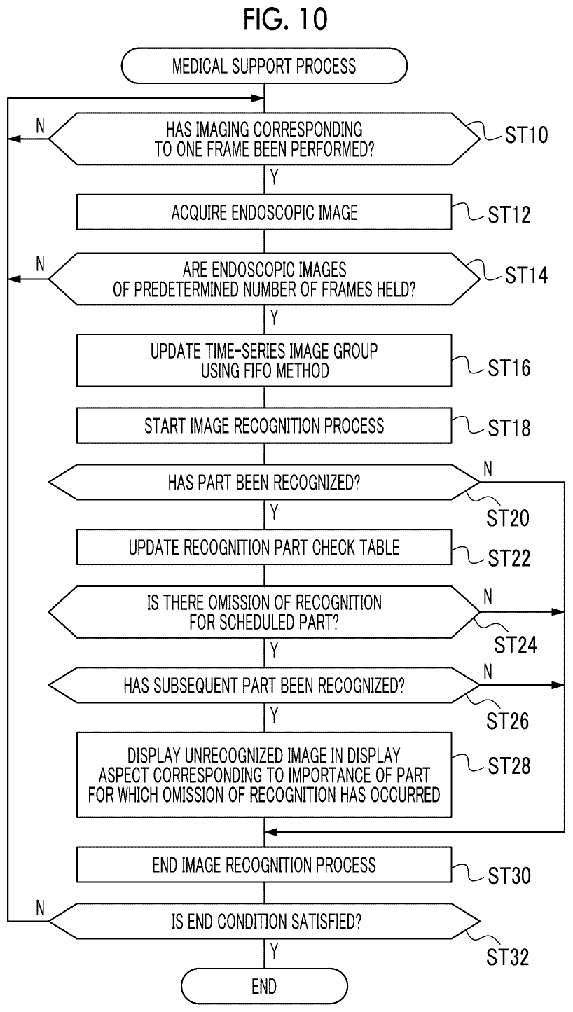

Therefore, in view of this circumstance, in the present embodiment, a medical support process is performed by the processor 70 of the control device 22 in order to suppress the omission of recognition by the image recognition process for a plurality of parts scheduled in advance, (see FIGS. 4 and 10). Further, in the present embodiment, the omission of the recognition is synonymous with the above-described omission of observation.

The medical support process includes a process that recognizes a plurality of parts in the observation target 21 on the basis of a plurality of endoscopic images 40 including the observation target 21 and outputs unrecognized information capable of specifying that an unrecognized part (that is, a part that has not been recognized by the processor 70) is present in a case where the unrecognized part in the observation target 21 is present in the plurality of parts. Hereinafter, the medical support process will be described in more detail.

For example, as illustrated in FIG. 4, a medical support processing program 76 is stored in the NVM 74. The medical support processing program 76 is an example of a “program” according to the technology of the present disclosure. The processor 70 reads the medical support processing program 76 from the NVM 74 and executes the read medical support processing program 76 on the RAM 72. The processor 70 operates as an image acquisition unit 70A, a recognition unit 70B, a control unit 70C according to the medical support processing program 76 executed on the RAM 72 to implement the medical support process.

A trained model 78 is stored in the NVM 74. In the present embodiment, the recognition unit 70B performs an AI-type image recognition process as the image recognition process for object detection. The AI-type image recognition process by the recognition unit 70B means an image recognition process using the trained model 78. The trained model 78 is a mathematical model for object detection and is obtained by performing machine learning on a neural network in advance to optimize the neural network. Hereinafter, the image recognition process using the trained model 78 will be described as a process that is actively performed by the trained model 78. That is, in the following description, for convenience of explanation, the trained model 78 is considered as a function of performing a process on input information and outputting the result of the process.

A recognition part check table 80 and an importance table 82 are stored in the NVM 74. Both the recognition part check table 80 and the importance table 82 are used by the control unit 70C.

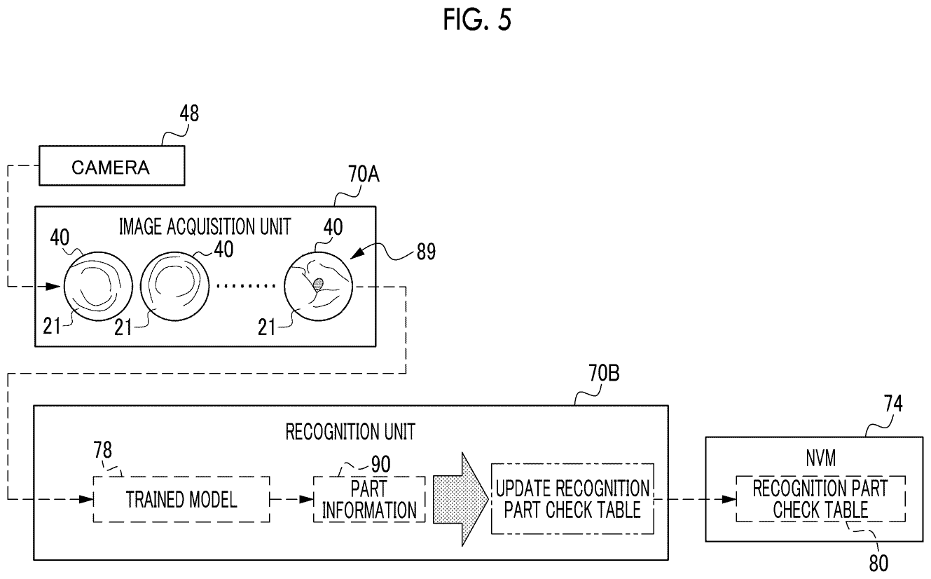

As illustrated in FIG. 5 as an example, the image acquisition unit 70A acquires the endoscopic images 40, which have been captured by the camera 48 at an imaging frame rate (for example, several tens of frames/sec) and generated, from the camera 48 frame by frame.

The image acquisition unit 70A holds a time-series image group 89. The time-series image group 89 is a plurality of time-series endoscopic images 40 including the observation target 21. The time-series image group 89 includes, for example, the endoscopic images 40 of a predetermined number of frames (for example, a predetermined number of frames within a range of several tens to several hundreds of frames). The image acquisition unit 70A updates the time-series image group 89 using a FIFO method each time the endoscopic image 40 is acquired from the camera 48.

Here, an example of the form in which the time-series image group 89 is held and updated by the image acquisition unit 70A has been described. However, this is only an example. For example, the time-series image group 89 may be held in a memory, such as the RAM 72, that is connected to the processor 70 and then updated.

The recognition unit 70B performs the image recognition process using the trained model 78 on the time-series image group 89 (that is, the plurality of time-series endoscopic images 40 held by the image acquisition unit 70A) to recognize the part of the observation target 21. In other words, the recognition of the part can be said to be the detection of the part. In the present embodiment, the recognition of the part indicates a process that specifies the name of the part and that stores the endoscopic image 40 including the recognized part and the name of the part included in the endoscopic image 40 in a memory (for example, the NVM 74 and/or an external storage device) to be associated with each other.

The trained model 78 is obtained by performing machine learning using first training data on the neural network to optimize the neural network. An example of the first training data is training data in which a plurality of images (for example, a plurality of images corresponding to a plurality of time-series endoscopic images 40) obtained in time series by imaging a part (for example, a part in the observation target 21) to be subjected to endoscopy are example data and part information 90 related to the part to be subjected to endoscopy is correct answer data. There are a plurality of parts such as a cardia, a fundus, a greater-curvature-side anterior wall of an upper gastric body, a greater-curvature-side posterior wall of the upper gastric body, a greater-curvature-side anterior wall of a middle gastric body, a greater-curvature-side posterior wall of the middle gastric body, a greater-curvature-side anterior wall of a lower gastric body, and a greater-curvature-side posterior wall of the lower gastric body. Machine learning is performed on the neural network using the first training data created for each part. The part information 90 includes information indicating the name of the part, coordinates that can specify the position of the part in the observation target 21, and the like.

In addition, here, a form in which only one trained model 78 is used by the recognition unit 70B is given as an example. However, this is only an example. For example, a trained model 78 selected from a plurality of trained models 78 may be used by the recognition unit 70B. In this case, each of the trained models 78 may be created by performing machine learning specialized for each type of endoscopy. The trained model 78 corresponding to the type of endoscopy that is currently being performed may be selected and used by the recognition unit 70B.

In the present embodiment, a trained model created by performing machine learning specialized for endoscopy for the stomach is applied as an example of the trained model 78 used by the recognition unit 70B.

In addition, here, an example of the form in which the trained model is created by performing machine learning specialized for endoscopy for the stomach on the neural network has been described. However, this is only an example. In a case where endoscopy is performed on a luminal organ other than the stomach, a trained model created by performing machine learning specialized for the type of the luminal organ, on which the endoscopy is performed, on the neural network may be used. An example of the luminal organ other than the stomach is the large intestine, the small intestine, the esophagus, the duodenum, or the bronchus. In addition, a trained model created by performing machine learning specialized for endoscopy for a plurality of luminal organs, such as the stomach, the large intestine, the small intestine, the esophagus, the duodenum, and the bronchus, on the neural network may be used as the trained model 78.

The recognition unit 70B performs the image recognition process using the trained model 78 on the time-series image group 89 acquired by the image acquisition unit 70A to recognize a plurality of parts included in the stomach (hereinafter, simply referred to as “a plurality of parts”). The plurality of parts are classified into major categories and minor categories included in the major categories. The “major category” referred to here is an example of a “major category” according to the technology of the present disclosure. In addition, the “minor category” referred to here is an example of a “minor category” according to the technology of the present disclosure.

The plurality of parts are roughly classified into the cardia, the fundus, a greater curvature of the upper gastric body, a greater curvature of the middle gastric body, a greater curvature of the lower gastric body, a greater curvature of a gastric angle, a greater curvature of an antrum, a duodenal bulb, a pyloric ring, a lesser curvature of the antrum, a lesser curvature of the gastric angle, a lesser curvature of the upper gastric body, a lesser curvature of the middle gastric body, and a lesser curvature of the lower gastric body as the major categories.

The greater curvature of the upper gastric body is classified into the greater-curvature-side anterior wall of the upper gastric body and the greater-curvature-side posterior wall of the upper gastric body as the minor categories. The greater curvature of the middle gastric body is classified into the greater-curvature-side anterior wall of the middle gastric body and the greater-curvature-side posterior wall of the middle gastric body as the minor categories. The greater curvature of the lower gastric body is classified into the greater-curvature-side anterior wall of the lower gastric body and the greater-curvature-side posterior wall of the lower gastric body as the minor categories. The greater curvature of the gastric angle is classified into the greater-curvature-side anterior wall of the gastric angle and the greater-curvature-side posterior wall of the gastric angle as the minor categories. The greater curvature of the antrum is classified into the greater-curvature-side anterior wall of the antrum and the greater-curvature-side posterior wall of the antrum as the minor categories. The lesser curvature of the antrum is classified into the lesser-curvature-side anterior wall of the antrum and the lesser-curvature-side posterior wall of the antrum as the minor categories. The lesser curvature of the gastric angle is classified into the lesser-curvature-side anterior wall of the gastric angle and the lesser-curvature-side posterior wall of the gastric angle as the minor categories. The lesser curvature of the lower gastric body is classified into the lesser-curvature-side anterior wall of the lower gastric body and the lesser-curvature-side posterior wall of the lower gastric body as the minor categories. The lesser curvature of the middle gastric body is classified into the lesser-curvature-side anterior wall of the middle gastric body and the lesser-curvature-side posterior wall of the middle gastric body as the minor categories. The lesser curvature of the upper gastric body is classified into the lesser-curvature-side anterior wall of the upper gastric body and the lesser-curvature-side posterior wall of the upper gastric body as the minor categories.

The recognition unit 70B acquires the time-series image group 89 from the image acquisition unit 70A and inputs the acquired time-series image group 89 to the trained model 78. Then, the trained model 78 outputs the part information 90 corresponding to the input time-series image group 89. The recognition unit 70B acquires the part information 90 output from the trained model 78.

The recognition part check table 80 is a table that is used to check whether or not the part scheduled to be recognized by the recognition unit 70B has been recognized. In the recognition part check table 80, the plurality of parts are associated with information indicating whether or not each part has been recognized by the recognition unit 70B. Since the name of the part is specified from the part information 90, the recognition unit 70B updates the recognition part check table 80 according to the part information 90 acquired from the trained model 78. That is, the recognition unit 70B updates the information (that is, information indicating whether or not the part has been recognized by the recognition unit 70B) corresponding to each part in the recognition part check table 80.

The control unit 70C displays the endoscopic image 40 acquired by the image acquisition unit 70A on the screen 36. The control unit 70C generates a detection frame 23 on the basis of the part information 90 and displays the generated detection frame 23 to be superimposed on the endoscopic image 40. The detection frame 23 is a frame that can specify the position of the part specified from the part information 90. For example, the detection frame 23 is generated on the basis of a bounding box that is used in the AI-type image recognition process. The detection frame 23 may be a rectangular frame that consists of a continuous line or a frame having a shape other than the rectangular shape. Further, for example, instead of the rectangular frame consisting of the continuous line, a frame that consists of discontinuous lines (that is, intermittent lines) may be used. In addition, for example, a plurality of marks that specify portions corresponding to four corners of the detection frame 23 may be displayed. Further, the part specified from the part information 90 may be filled with a predetermined color (for example, a translucent color).

In addition, here, an example of the form in which the AI-type process (for example, the process by the recognition unit 70B) is performed by the control device 22 has been described. However, the technology of the present disclosure is not limited thereto. For example, the AI-type process may be performed by a device that is separate from the control device 22. In this case, for example, the device that is separate from the control device 22 acquires the endoscopic image 40 and various parameters used to observe the observation target 21 with the endoscope 12 and outputs an image obtained by superimposing the detection frame 23 and/or various maps (for example, the medical support image 41) on the endoscopic image 40 to the display device 13 and the like.

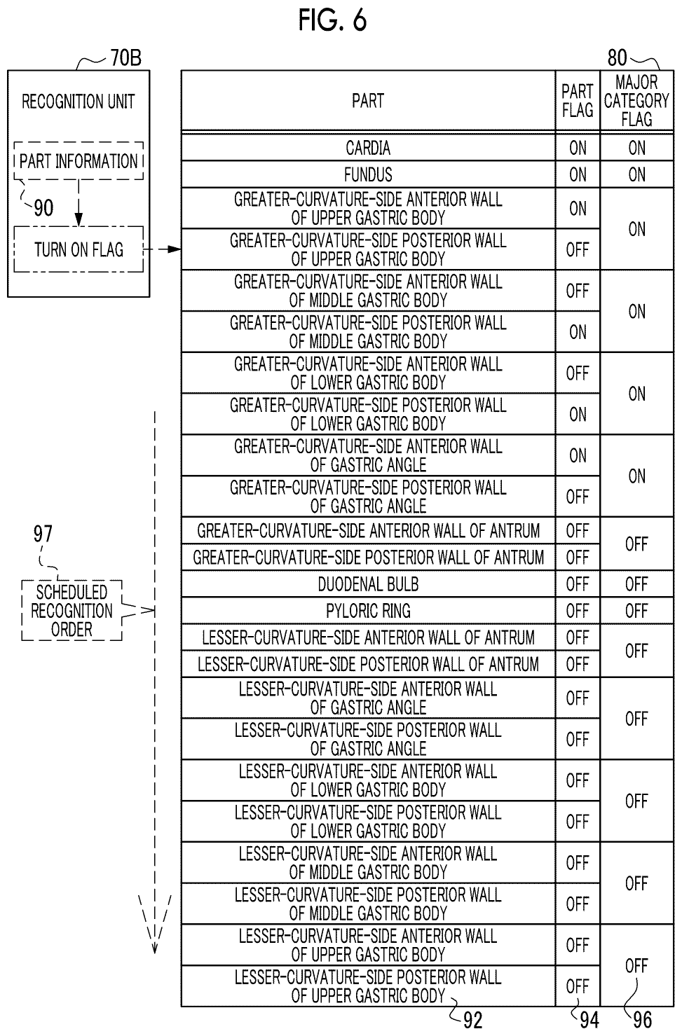

For example, as illustrated in FIG. 6, the recognition part check table 80 is a table in which a part name 92 is associated with a part flag 94 and a major category flag 96. The part name 92 is the name of a part. In the recognition part check table 80, a plurality of part names 92 are arranged in a scheduled recognition order 97. The scheduled recognition order 97 indicates the order of the parts scheduled to be recognized by the recognition unit 70B. The part scheduled to be recognized by the recognition unit 70B is an example of a “scheduled part” according to the technology of the present disclosure, and the scheduled recognition order 97 is an example of a “second order” according to the technology of the present disclosure.

The part flag 94 is a flag indicating whether or not the part corresponding to the part name 92 has been recognized by the recognition unit 70B. The part flag 94 is switched between on (for example, 1) and off (for example, 0). The part flag 94 is off as a default. In a case where the part corresponding to the part name 92 is recognized, the recognition unit 70B turns on the part flag 94 corresponding to the part name 92 indicating the recognized part.

The major category flag 96 is a flag indicating whether or not the part corresponding to the major category has been recognized by the recognition unit 70B. The major category flag 96 is switched between on (for example, 1) and off (for example, 0). The major category flag 96 is off as a default. In a case where the recognition unit 70B recognizes a part classified into the major category (for example, a part classified into the minor category among the parts classified into the major category), that is, a part corresponding to the part name 92, the major category flag 96 corresponding to the major category into which the recognized portion is classified is turned on. In other words, in a case where the part flag 94 corresponding to the major category flag 96 is turned on, the major category flag 96 is turned on.

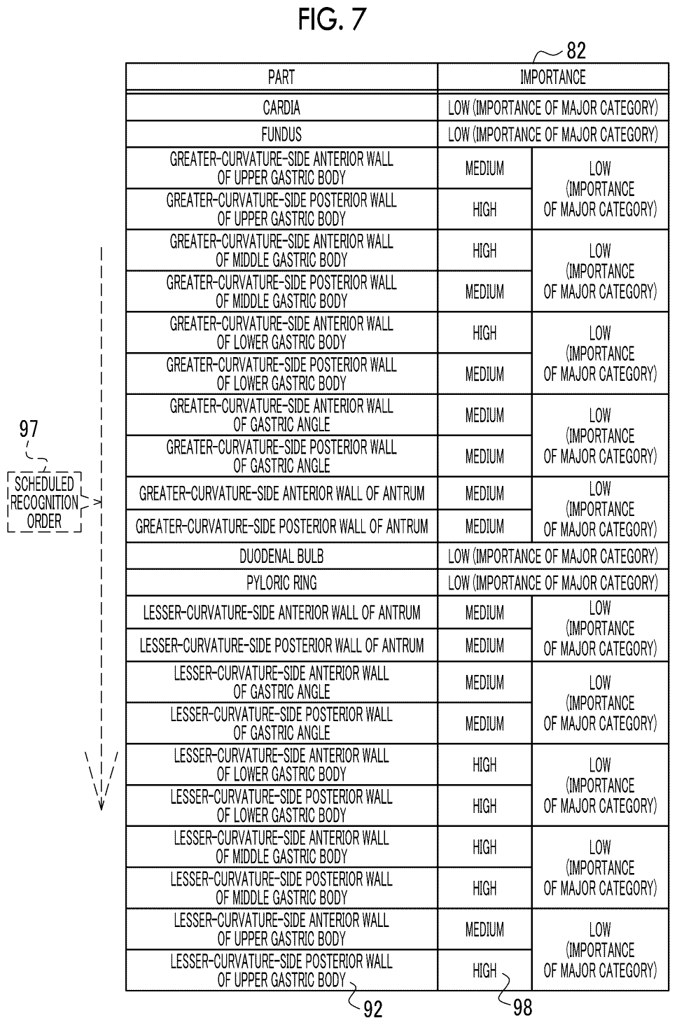

For example, as illustrated in FIG. 7, the importance table 82 is a table in which importance 98 is associated with the part name 92. That is, the importance 98 is assigned to a plurality of parts. The importance 98 is an example of “importance” according to the technology of the present disclosure.

In the importance table 82, a plurality of part names 92 are arranged in the order of the parts scheduled to be recognized by the recognition unit 70B. That is, in the importance table 82, the plurality of part names 92 are arranged in the scheduled recognition order 97. The importance 98 is the importance of the part specified from the part name 92. The importance 98 is defined by any one of three levels of a “high” level, a “medium” level, and a “low” level. The “high” level or the “medium” level is given as the importance 98 to the part classified into the minor category, and the “low” level is given as the importance 98 to the part classified into the major category.

In the example illustrated in FIG. 7, the “high” level is given as the importance 98 to the greater-curvature-side posterior wall of the upper gastric body, the greater-curvature-side anterior wall of the middle gastric body, the greater-curvature-side anterior wall of the lower gastric body, the lesser-curvature-side anterior wall of the lower gastric body, the lesser-curvature-side posterior wall of the lower gastric body, the lesser-curvature-side anterior wall of the middle gastric body, the lesser-curvature-side posterior wall of the middle gastric body, and the lesser-curvature-side posterior wall of the upper gastric body.

The “medium” level is given as the importance 98 to each part classified into the minor category other than the greater-curvature-side posterior wall of the upper gastric body, the greater-curvature-side anterior wall of the middle gastric body, the greater-curvature-side anterior wall of the lower gastric body, the lesser-curvature-side anterior wall of the lower gastric body, the lesser-curvature-side posterior wall of the lower gastric body, the lesser-curvature-side anterior wall of the middle gastric body, the lesser-curvature-side posterior wall of the middle gastric body, and the lesser-curvature-side posterior wall of the upper gastric body. That is, the “medium” level is given as the importance 98 to the greater-curvature-side anterior wall of the upper gastric body, the greater-curvature-side posterior wall of the middle gastric body, the greater-curvature-side posterior wall of the lower gastric body, the greater-curvature-side anterior wall of the gastric angle, the greater-curvature-side posterior wall of the gastric angle, the greater-curvature-side anterior wall of the antrum, the greater-curvature-side posterior wall of the antrum, the lesser-curvature-side anterior wall of the antrum, the lesser-curvature-side posterior wall of the antrum, the lesser-curvature-side anterior wall of the gastric angle, the lesser-curvature-side posterior wall of the gastric angle, and the lesser-curvature-side anterior wall of the upper gastric body.

Each part classified into the major categories, such as the cardia, the fundus, the greater curvature of the upper gastric body, the greater curvature of the middle gastric body, the greater curvature of the lower gastric body, the greater curvature of the gastric angle, the greater curvature of the antrum, the duodenal bulb, the pyloric ring, the lesser curvature of the antrum, the lesser curvature of the gastric angle, the lesser curvature of the upper gastric body, the lesser curvature of the middle gastric body, and the lesser curvature of the lower gastric body has lower importance 98 than the part classified into the minor category. In the example illustrated in FIG. 7, the “low” level is given as the importance 98 to the cardia, the fundus, the greater curvature of the upper gastric body, the greater curvature of the middle gastric body, the greater curvature of the lower gastric body, the greater curvature of the gastric angle, the greater curvature of the antrum, the duodenal bulb, the pyloric ring, the lesser curvature of the antrum, the lesser curvature of the gastric angle, the lesser curvature of the upper gastric body, the lesser curvature of the middle gastric body, and the lesser curvature of the lower gastric body. In other words, the part classified into the minor category may be given higher importance 98 than the part classified into the major category.

The “high”, “medium”, and “low” levels of the importance 98 are determined in response to an instruction given from the outside to the endoscope 12. The receiving device 62 is given as an example of a first unit that gives an instruction for the importance 98 to the endoscope 12. In addition, a communication device (for example, a tablet terminal, a personal computer, and/or a server) that is connected to the endoscope 12 such that it can communicate therewith) is given as an example of a second unit that gives an instruction for the importance 98 to the endoscope 12.

In addition, the importance 98 associated with the plurality of part names 92 is determined according to the data of a past examination (for example, statistical data based on the data of the past examination obtained from a plurality of subjects 20) performed on a plurality of parts.

For example, the importance 98 corresponding to a part which is determined to be a part for which the omission of recognition is typically likely to occur, among a plurality of parts, is set to be higher than the importance 98 corresponding to a part which is determined to be a part for which the omission of recognition is typically unlikely to occur, among the plurality of parts. Whether or not the omission of recognition is typically likely to occur is derived from the data of the past examination performed on a plurality of parts by, for example, a statistical method. In the present embodiment, the “high” importance 98 indicates that the probability that the omission of recognition will typically occur is high. In addition, the “medium” importance 98 indicates that the probability that the omission of recognition will typically occur is medium. Further, the “low” importance 98 indicates that the probability that the omission of recognition will typically occur is low.

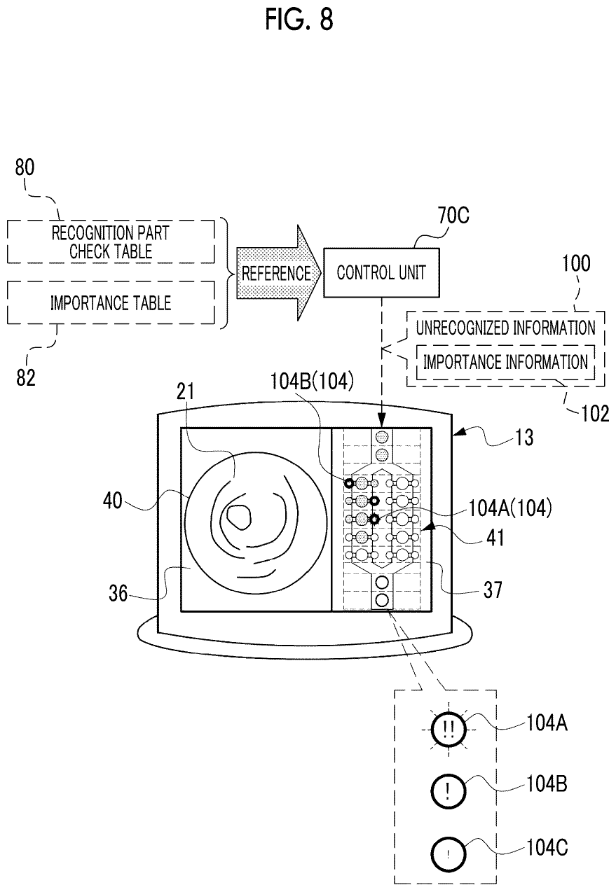

As illustrated in FIG. 8 as an example, the control unit 70C outputs unrecognized information 100 in a case in which an unrecognized part of the observation target 21 is present among a plurality of parts according to the recognition part check table 80 and the importance table 82. The unrecognized information 100 is information that can specify that the unrecognized part is present. The unrecognized information 100 includes importance information 102. The importance information 102 is information that can specify the importance 98 obtained from the importance table 82.

The output destination of the unrecognized information 100 is the display device 13. However, this is only an example, and the output destination of the unrecognized information 100 may be, for example, a tablet terminal, a personal computer, and/or a server that is connected to the endoscope 12 such that it can communicate therewith.

The unrecognized information 100 is displayed as the medical support image 41 on the screen 37 by the control unit 70C. The medical support image 41 is an example of a “schematic view” and a “first schematic view” according to the technology of the present disclosure. The control unit 70C displays the importance information 102 included in the unrecognized information 100 as an importance mark 104 in the medical support image 41.

The display aspect of the importance mark 104 differs depending on the importance information 102. The importance marks 104 are classified into a first importance mark 104A, a second importance mark 104B, and a third importance mark 104C. The first importance mark 104A is a mark representing “high” importance 98. The second importance mark 104B is a mark representing “medium” importance 98. The third importance mark 104C is a mark representing “low” importance 98. That is, the first importance mark 104A, the second importance mark 104B, and the third importance mark 104C are marks that are represented in a display aspect in which the “high”, “medium”, and “low” levels of importance can be distinguished. The second importance mark 104B is displayed in a state in which it is emphasized more than the third importance mark 104C, and the first importance mark 104A is displayed in a state in which it is emphasized more than the second importance mark 104B.

In the example illustrated in FIG. 8, the first importance mark 104A includes a plurality of exclamation marks (here, for example, two exclamation marks), and each of the second importance mark 104B and the third importance mark 104C includes one exclamation mark. The size of the exclamation mark included in the third importance mark 104C is smaller than the size of the exclamation mark included in the first importance mark 104A and the second importance mark 104B. In addition, the second importance mark 104B is colored to be more conspicuous than the third importance mark 104C, and the first importance mark 104A is colored to be more conspicuous than the second importance mark 104B. Further, the brightness of the second importance mark 104B is higher than the brightness of the third importance mark 104C, and the brightness of the first importance mark 104A is higher than the brightness of the second importance mark 104B. As described above, as a relationship of the degree of conspicuousness, a relationship of “the first importance mark 104A>the second importance mark 104B>the third importance mark 104C” is established.

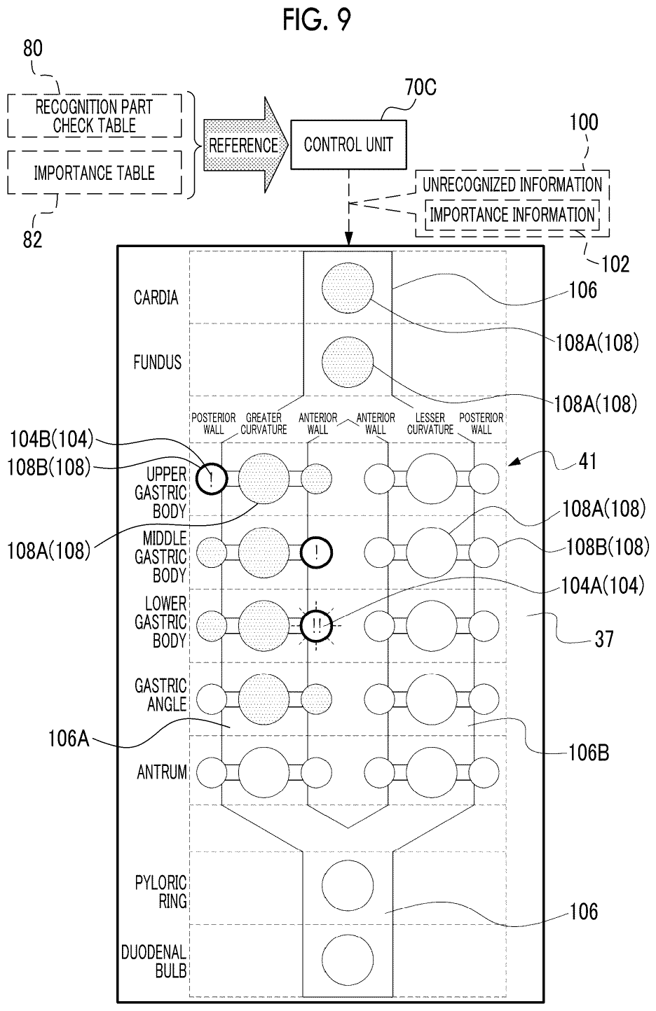

As illustrated in FIG. 9 as an example, the medical support image 41 includes a route 106. The route 106 is a route that schematically represents the order in which the stomach is observed using the endoscope 12 (here, for example, the scheduled recognition order 97 (see FIG. 6 and FIG. 7)) and is a schematic view in which the observation target 21 is divided into a plurality of regions corresponding to a plurality of parts. In the example illustrated in FIG. 9, as an example of the plurality of regions, in the medical support image 41, the cardia, the fundus, the upper gastric body, the middle gastric body, the lower gastric body, the gastric angle, the antrum, the pyloric ring, and the duodenal bulb are displayed in text, and the route 106 is also divided into the cardia, the fundus, the upper gastric body, the middle gastric body, the lower gastric body, the gastric angle, the antrum, the pyloric ring, and the duodenal bulb.

The route 106 is branched into a greater-curvature-side route 106A and a lesser-curvature-side route 106B in the middle from the most upstream side to the downstream side of the stomach, and the branched routes are joined. On the route 106, a large circular mark 108A is assigned to the part classified into the major category, and a small circular mark 108B is assigned to the part classified into the minor category. Hereinafter, for convenience of explanation, in a case where the circular marks 108A and 108B do not need to be distinguished from each other for description, they are referred to as “circular marks 108”.

In a portion of the route 106 from the most upstream side of the stomach to the front of the branch point of the greater-curvature-side route 106A and the lesser-curvature-side route 106B, the circular mark 108A corresponding to the cardia and the circular mark 108A corresponding to the fundus are arranged from the most upstream side of the stomach to the downstream side of the stomach.

In the greater-curvature-side route 106A, the circular mark 108A corresponding to the greater curvature, the circular mark 108B corresponding to the anterior wall, and the circular mark 108B corresponding to the posterior wall are disposed in units of the parts classified into the major categories. The circular mark 108A corresponding to the greater curvature is located at the center of the greater-curvature-side route 106A, and the circular mark 108B corresponding to the anterior wall and the circular mark 108B corresponding to the posterior wall are located on the left and right sides of the circular mark 108A corresponding to the greater curvature.

In the lesser-curvature-side route 106B, the circular mark 108A corresponding to the lesser curvature, the circular mark 108B corresponding to the anterior wall, and the circular mark 108B corresponding to the posterior wall are disposed in units of the parts classified into the major categories. The circular mark 108A corresponding to the lesser curvature is located at the center of the lesser-curvature-side route 106B, and the circular mark 108B corresponding to the anterior wall and the circular mark 108B corresponding to the posterior wall are located on the left and right sides of the circular mark 108A corresponding to the lesser curvature.

In a portion of the route 106 from a junction point of the greater-curvature-side route 106A and the lesser-curvature-side route 106B to a part on the most downstream side of the stomach, the circular mark 108A corresponding to the pyloric ring and the circular mark 108A corresponding to the duodenal bulb are arranged.

The inside of the circular mark 108 is blank as a default. In a case where the part corresponding to the circular mark 108 is recognized by the recognition unit 70B, the inside of the circular mark 108 corresponding to the portion recognized by the recognition unit 70B is filled with a specific color (for example, a predetermined color among three primary colors of light and three primary colors of pigment). On the other hand, in a case in which the part corresponding to the circular mark 108 has not been recognized by the recognition unit 70B, the inside of the circular mark 108 corresponding to the part which has not been recognized by the recognition unit 70B is not filled with any color. However, the importance mark 104 corresponding to the importance 98 of the part which has not been recognized by the recognition unit 70B is displayed in the circular mark 108 corresponding to the part which has not been recognized by the recognition unit 70B. As described above, the circular mark 108 corresponding to the part that has been recognized by the recognition unit 70B and the circular mark 108 corresponding to the part that has not been recognized by the recognition unit 70B are displayed in the medical support image 41 on the display device 13 in an aspect in which the circular marks 108 can be distinguished from each other.

In addition, the image obtained by filling the circular mark 108 with the specific color is an example of a “second image capable of specifying a part other than an unrecognized part among a plurality of parts” according to the technology of the present disclosure. The image obtained by displaying the importance mark 104 corresponding to the importance 98 of the part in the circular mark 108 is an example of a “first image capable of specifying an unrecognized part” according to the technology of the present disclosure.

The control unit 70C updates the content of the medical support image 41 in a case in which the major category flag 96 in the recognition part check table 80 is turned on. The update of the content of the medical support image 41 is achieved by the output of the unrecognized information 100 by the control unit 70C.

In a case where the major category flag 96 in the recognition part check table 80 is turned on, the control unit 70C fills the circular mark 108A of the part corresponding to the turned-on major category flag 96 with a specific color. In addition, in a case where the part flag 94 is turned on, the control unit 70C fills the circular mark 108B of the part corresponding to the turned-on part flag 94 with a specific color.

Further, in a case where a plurality of minor categories are included in the major category and the part flag 94 corresponding to the part classified into one minor category is turned on, the major category flag 96 corresponding to the part which is classified into the minor category having the turned-on part flag 94 is turned on.

On the other hand, in a case in which a part has not been recognized by the recognition unit 70B, the control unit 70C displays the importance mark 104 in the circular mark 108 corresponding to the part that has not been recognized by the recognition unit 70B on condition that the recognition unit 70B recognizes a subsequent part scheduled to be recognized by the recognition unit 70B after the part that has not been recognized by the recognition unit 70B. That is, in a case in which it is confirmed that the order of the parts recognized by the recognition unit 70B deviates from the scheduled recognition order 97 (FIGS. 6 and 7), the control unit 70C displays the importance mark 104 in the circular mark 108 corresponding to the part that has not been recognized by the recognition unit 70B. The reason for doing so is to notify of the omission of recognition by the recognition unit 70B at the timing when the omission of recognition by the recognition unit 70B is confirmed (for example, the timing when the probability that the doctor 14 will forget to observe a part during the process of operating the endoscope 12 is very high). In addition, here, the order recognized by the recognition unit 70B is an example of a “first order” according to the technology of the present disclosure.

Here, an example of the subsequent part that is scheduled to be recognized after the part which has not been recognized by the recognition unit 70B is a part that is classified into the major category scheduled to be recognized immediately after the major category into which the part which has not been recognized by the recognition unit 70B is classified. Here, the major category into which the part which has not been recognized by the recognition unit 70B is classified is an example of a “first major category” according to the technology of the present disclosure. In addition, the major category scheduled to be recognized immediately after the major category into which the part which has not been recognized by the recognition unit 70B is classified is an example of a “second major category” according to the technology of the present disclosure.

In the example illustrated in FIG. 9, in a case where the greater-curvature-side posterior wall of the upper gastric body has not been recognized by the recognition unit 70B, the second importance mark 104B is displayed to be superimposed on the circular mark 108B corresponding to the greater-curvature-side posterior wall of the upper gastric body on condition that the recognition unit 70B recognizes a part that is classified into the major category scheduled to be recognized immediately after the major category into which the greater-curvature-side posterior wall of the upper gastric body is classified. Here, the major category into which the greater-curvature-side posterior wall of the upper gastric body is classified indicates the greater curvature of the upper gastric body. In addition, the major category scheduled to be recognized immediately after the major category into which the greater-curvature-side posterior wall of the upper gastric body is classified indicates the greater curvature of the middle gastric body.

In addition, in the example illustrated in FIG. 9, in a case where the greater-curvature-side anterior wall of the middle gastric body has not been recognized by the recognition unit 70B, the second importance mark 104B is displayed to be superimposed on the circular mark 108B corresponding to the greater-curvature-side anterior wall of the middle gastric body on condition that the recognition unit 70B recognizes a part that is classified into the major category scheduled to be recognized immediately after the major category into which the greater-curvature-side anterior wall of the middle gastric body is classified. Here, the major category into which greater-curvature-side anterior wall of the middle gastric body is classified indicates the greater curvature of the middle gastric body. In addition, the major category scheduled to be recognized immediately after the major category into which the greater-curvature-side anterior wall of the middle gastric body is classified indicates the greater curvature of the lower gastric body.

Further, in the example illustrated in FIG. 9, in a case where the greater-curvature-side anterior wall of the lower gastric body has not been recognized by the recognition unit 70B, the first importance mark 104A is displayed to be superimposed on the circular mark 108B corresponding to the greater-curvature-side anterior wall of the lower gastric body on condition that the recognition unit 70B recognizes a part that is classified into the major category scheduled to be recognized immediately after the major category into which the greater-curvature-side anterior wall of the lower gastric body is classified. Here, the major category into which the greater-curvature-side anterior wall of the lower gastric body is classified indicates the greater curvature of the lower gastric body. In addition, the major category scheduled to be recognized immediately after the major category into which the greater-curvature-side anterior wall of the lower gastric body is classified indicates the greater curvature of the gastric angle.

In the present embodiment, the image obtained by superimposing the importance mark 104 on the circular mark 108 is displayed in a state in which it is emphasized more than the image obtained by filling the circular mark 108 with a specific color in order to facilitate the specification of the part which has not been recognized by the recognition unit 70B. In the example illustrated in FIG. 9, an edge of the image obtained by superimposing the importance mark 104 on the circular mark 108 is displayed in a state in which it is enhanced more than an edge of the image obtained by filling the circular mark 108 with a specific color. The enhancement of the edge is achieved, for example, by adjusting the brightness of the edge. In addition, while the image obtained by filling the circular mark 108 with a specific color does not include the exclamation mark, the image obtained by superimposing the importance mark 104 on the circular mark 108 includes the exclamation mark. Therefore, the part that has not been recognized by the recognition unit 70B and the part that has been recognized by the recognition unit 70B are visually specified depending on whether the exclamation mark is present or absent.

Next, the operation of a portion of the endoscope system 10 according to the technology of the present disclosure will be described with reference to FIG. 10.

FIG. 10 illustrates an example of a flow of the medical support process performed by the processor 70. The flow of the medical support process illustrated in FIG. 10 is an example of the “medical support method” according to the technology of the present disclosure.

In the medical support process illustrated in FIG. 10, first, in Step ST10, the image acquisition unit 70A determines whether or not imaging corresponding to one frame has been performed by the camera 48. In a case in which the imaging corresponding to one frame has not been performed by the camera 48 in Step ST10, the determination result is “No”, and the determination in Step ST10 is performed again. In a case where the imaging corresponding to one frame has been performed by the camera 48 in Step ST10, the determination result is “Yes”, and the medical support process proceeds to Step ST12.

In Step ST12, the image acquisition unit 70A acquires the endoscopic image 40 of one frame from the camera 48. After the process in Step ST12 is executed, the medical support process proceeds to Step ST14.

In Step ST14, the image acquisition unit 70A determines whether or not the endoscopic images 40 of a predetermined number of frames are held. In a case where the endoscopic images 40 of the predetermined number of frames are not held in Step ST14, the determination result is “No”, and the medical support process proceeds to Step ST10. In a case where the endoscopic images 40 of the predetermined number of frames are held in Step ST14, the determination result is “Yes”, and the medical support process proceeds to Step ST16.

In Step ST16, the image acquisition unit 70A adds the endoscopic image 40 acquired in Step ST12 to the time-series image group 89 using the FIFO method to update the time-series image group 89. After the process in Step ST16 is executed, the medical support process proceeds to Step ST18.

In Step ST18, the recognition unit 70B starts the execution of the AI-type image recognition process (that is, the image recognition process using the trained model 78) on the time-series image group 89 updated in Step ST16. After the process in Step ST18 is executed, the medical support process proceeds to Step ST20.

In Step ST20, the recognition unit 70B determines whether or not any of a plurality of parts in the observation target 21 has been recognized. In a case where the recognition unit 70B has not recognized any of the plurality of parts in the observation target 21 in Step ST20, the determination result is “No”, and the medical support process proceeds to Step ST30. In a case where the recognition unit 70B has recognized any of the plurality of parts in the observation target 21 in Step ST20, the determination result is “Yes”, and the medical support process proceeds to Step ST22.

In Step ST22, the recognition unit 70B updates the recognition part check table 80. That is, the recognition unit 70B turns on the part flag 94 and the major category flag 96 corresponding to the recognized part to update the recognition part check table 80. After the process in Step ST22 is executed, the medical support process proceeds to Step ST24.

In Step ST24, the control unit 70C determines whether or not the omission of recognition has occurred for the part scheduled in advance to be recognized by the recognition unit 70B. The determination of whether or not the omission of recognition has occurred is achieved, for example, by determining whether or not the order of the parts recognized by the recognition unit 70B deviates from the scheduled recognition order 97. In a case where the omission of recognition has occurred for the part scheduled in advance to be recognized by the recognition unit 70B in Step ST24, the determination result is “Yes”, and the medical support process proceeds to Step ST26. In a case where the omission of recognition has not occurred for the part scheduled in advance to be recognized by the recognition unit 70B in Step ST24, the determination result is “No”, and the medical support process proceeds to Step ST30.

In a case in which the determination result is “No” in Step ST24 in a state in which the medical support image 41 is not displayed on the screen 37, the control unit 70C displays the medical support image 41 on the screen 37 and fills the circular mark 108 corresponding to the part recognized by the recognition unit 70B with a specific color. In addition, in a case where the determination result is “No” in Step ST24 in a state in which the medical support image 41 is displayed on the screen 37, the control unit 70C updates the content of the medical support image 41. That is, the control unit 70C fills the circular mark 108 corresponding to the part recognized by the recognition unit 70B with a specific color. Therefore, the doctor 14 visually ascertains which part has been recognized by the recognition unit 70B from the medical support image 41 displayed on the screen 37.

In Step ST26, the control unit 70C determines whether or not a part subsequent to the part not recognized by the recognition unit 70B has been recognized by the recognition unit 70B. The part subsequent to the part not recognized by the recognition unit 70B indicates, for example, a part that is classified into a major category scheduled to be recognized by the recognition unit 70B immediately after the major category into which the part not recognized by the recognition unit 70B is classified. In a case in which the part subsequent to the part not recognized by the recognition unit 70B has not been recognized by the recognition unit 70B in Step ST26, the determination result is “No”, and the medical support process proceeds to Step ST30. In a case in which the part subsequent to the part not recognized by the recognition unit 70B has been recognized by the recognition unit 70B in Step ST26, the determination result is “Yes”, and the medical support process proceeds to Step ST28.

In Step ST28, the control unit 70C displays the unrecognized image in the medical support image 41 in a display aspect corresponding to the importance 98 of the part for which the omission of recognition has occurred, with reference to the importance table 82. That is, the control unit 70C displays the importance mark 104 corresponding to the importance 98 of the part for which the omission of recognition has occurred, to be superimposed on the circular mark 108. The first importance mark 104A, the second importance mark 104B, and the third importance mark 104C are selectively displayed to be superimposed on the circular mark 108 according to the importance 98 corresponding to the part for which the omission of recognition has occurred. Therefore, the doctor 14 visually ascertains which part has not been recognized by the recognition unit 70B, and the importance 98 assigned to the part is visually distinguishable. After the process in Step ST28 is executed, the medical support process proceeds to Step ST30.

In Step ST30, the recognition unit 70B ends the execution of the AI-type image recognition process on the time-series image group 89. After the process in Step ST30 is executed, the medical support process proceeds to Step ST32.

In Step ST32, the control unit 70C determines whether or not a medical support process end condition is satisfied. An example of the medical support process end condition is a condition that an instruction for the endoscope system 10 to end the medical support process is given (for example, a condition that the receiving device 62 receives an instruction to end the medical support process).

In a case where the medical support process end condition is not satisfied in Step ST32, the determination result is “No”, and the medical support process proceeds to Step ST10 illustrated in FIG. 10. In a case where the medical support process end condition is satisfied in Step ST32, the determination result is “Yes”, and the medical support process ends.

As described above, in the endoscope system 10, the plurality of parts are recognized by the recognition unit 70B by repeatedly executing the process in Step ST10 to the process in Step ST32 in the medical support process. Then, in a case where the unrecognized part (that is, the part that has not been recognized by the recognition unit 70B) is present in the plurality of parts in the observation target 21 (here, for example, the stomach), the control unit 70C outputs the unrecognized information 100 to the display device 13. The unrecognized information 100 is displayed as the medical support image 41 on the screen 37. In the medical support image 41, the unrecognized part is displayed as the importance mark 104. This enables the doctor 14 to visually ascertain where the unrecognized part is. Therefore, the doctor 14 can retry the imaging of the unrecognized part using the camera 48 while referring to the medical support image 41. In a case where the recognition unit 70B performs the AI-type image recognition process on the endoscopic image 40 obtained by retrying the imaging of the unrecognized part again, it is possible to recognize the part that could not be recognized before. As described above, according to the endoscope system 10, it is possible to contribute to suppressing the omission of the recognition for the part in the observation target 21.

In addition, in the endoscope system 10, in a case where the part has not been recognized by the recognition unit 70B, the control unit 70C outputs the unrecognized information 100 to the display device 13 on condition that a subsequent part that is scheduled to be recognized by the recognition unit 70B after the unrecognized part is recognized. For example, in a case where the part has not been recognized by the recognition unit 70B, the control unit 70C outputs the unrecognized information 100 to the display device 13 on condition that the part classified into the major category that is scheduled to be recognized by the recognition unit 70B after the unrecognized part is recognized. Therefore, according to the endoscope system 10, in a situation in which there is a high probability that the omission of recognition for the part in the observation target 21 will occur, the doctor 14 can ascertain that the omission of recognition for the part in the observation target 21 has occurred.

In addition, in the endoscope system 10, the unrecognized information 100 is output to the display device 13 on the basis of the order in which a plurality of parts are recognized by the recognition unit 70B and the scheduled recognition order 97. That is, in a case where the order in which the plurality of parts are recognized by the recognition unit 70B deviates from the scheduled recognition order 97, the unrecognized information 100 is output to the display device 13. Therefore, it is possible to easily specify whether or not the part in the observation target 21 is the unrecognized part.

In addition, in the endoscope system 10, the unrecognized information 100 output from the control unit 70C includes the importance information 102, and the importance information 102 is displayed as the importance mark 104 in the medical support image 41. Therefore, the doctor 14 can visually ascertain the importance 98 of the unrecognized part.

In addition, in the endoscope system 10, the importance 98 assigned to the part is determined in response to an instruction given from the outside. Therefore, it is possible to suppress the omission of the recognition of a part with high importance 98 determined in response to the instruction given from the outside among a plurality of parts.

In addition, in the endoscope system 10, the importance 98 assigned to the part is determined according to the data of the past examination performed on a plurality of parts. Therefore, it is possible to suppress the omission of the recognition of a part with high importance 98 determined according to the data of the past examination.

In addition, in the endoscope system 10, the importance 98 corresponding to a part which is determined to be a part for which the omission of recognition is typically likely to occur, among a plurality of parts, is set to be higher than the importance 98 corresponding to a part which is determined to be a part for which the omission of recognition is typically unlikely to occur, among the plurality of parts. Therefore, it is possible to suppress the omission of the recognition of the part, which is determined to be the part for which the omission of recognition is typically likely to occur, among the plurality of parts.

In addition, in the endoscope system 10, higher importance 98 is assigned to the part classified into the minor category than the part classified into the major category. Therefore, it is possible to suppress the omission of the recognition of the part classified into the minor category, as compared to a case where the same level of importance 98 is assigned to the part classified into the major category and the part classified into the minor category.

In addition, in the endoscope system 10, the medical support image 41 is displayed on the screen 37. Then, the image obtained by filling the circular mark 108 with a specific color and the image obtained by displaying the importance mark 104 to be superimposed on the circular mark 108 are displayed in the medical support image 41. The image obtained by filling the circular mark 108 with the specific color is an image corresponding to the part that has been recognized by the recognition unit 70B, and the image obtained by displaying the importance mark 104 to be superimposed on the circular mark 108 is an image corresponding to the part that has not been recognized by the recognition unit 70B. Therefore, the doctor 14 can visually ascertain the unrecognized part and the part (that is, the part that has been recognized by the recognition unit 70B) other than the unrecognized part from the medical support image 41 displayed on the screen 37.

In addition, in the endoscope system 10, the medical support image 41 is displayed on the screen 37. The medical support image 41 is a schematic view and includes the route 106. The route 106 is a route that represents the scheduled recognition order 97 and is a schematic view in which the observation target 21 is divided into a plurality of regions corresponding to a plurality of parts. Therefore, the doctor 14 can easily ascertain the positional relationship between the unrecognized part and the part other than the unrecognized part in the observation target 21.

In addition, in the endoscope system 10, the medical support image 41 is displayed on the screen 37. Then, the image obtained by filling the circular mark 108 with a specific color and the image obtained by displaying the importance mark 104 to be superimposed on the circular mark 108 are displayed in the medical support image 41. The image obtained by displaying the importance mark 104 to be superimposed on the circular mark 108 is displayed in a state in which it is emphasized more than the image obtained by filling the circular mark 108 with a specific color. Therefore, the doctor 14 can easily perceive the omission of the recognition of the part.

Further, in the endoscope system 10, the display aspect of the importance mark 104 that is displayed to be superimposed on the circular mark 108 differs depending on the importance 98 assigned to a plurality of parts. Therefore, the degree of attention of the doctor 14 to the unrecognized part can differ depending on the importance 98 assigned to the unrecognized part.

In addition, in the above-described embodiment, an example of the form in which the screens 36 and 37 are displayed on the display device 13 to be comparable has been described. However, this is only an example, and the screen 36 and the screen 37 may be selectively displayed. Further, the size ratio of the screen 36 to the screen 37 may be changed according to, for example, the instruction received by the receiving device 62 and/or the current state of the endoscope 12 (for example, the operation state of the endoscope 12).

In the above-described embodiment, an example of the form in which the recognition unit 70B performs the AI-type image recognition process has been described. However, the technology of the present disclosure is not limited thereto. For example, the recognition unit 70B may perform a non-AI-type (for example, template-matching-type) image recognition process to recognize the part. Further, the recognition unit 70B may recognize the part using both the AI-type image recognition process and the non-AI-type image recognition process.

In the above-described embodiment, an example of the form in which the recognition unit 70B performs the image recognition process on the time-series image group 89 to recognize a part has been described. However, this is only an example, and the image recognition process may be performed on the endoscopic image 40 of a single frame to recognize a part.

In the above-described embodiment, the recognition unit 70B performs the image recognition process on condition that the time-series image group 89 is updated. However, the technology of the present disclosure is not limited thereto. For example, the recognition unit 70B may perform the image recognition process on condition that a specific instruction (for example, an instruction for the recognition unit 70B to start the image recognition process) is given from the doctor 14 to the endoscope 12 via a communication device that is connected to the receiving device 62 or the endoscope 12 such that it can communicate therewith.

In the above-described embodiment, the display aspect of the first importance mark 104A, the display aspect of the second importance mark 104B, and the display aspect of the third importance mark 104C are different depending on the importance 98. However, the technology of the present disclosure is not limited thereto. For example, the display aspect of the first importance mark 104A, the display aspect of the second importance mark 104B, and the display aspect of the third importance mark 104C may be different depending on the type of the unrecognized part. For example, the display aspect of the importance mark 104 displayed to be superimposed on the circular mark 108B corresponding to the greater-curvature-side posterior wall of the upper gastric body and the display aspect of the importance mark 104 displayed to be superimposed on the circular mark 108B corresponding to the greater-curvature-side anterior wall of the middle gastric body may be different to be distinguishable from each other. This enables the doctor 14 to visually ascertain the type of the unrecognized part.

In addition, even in a case where the display aspect of the importance mark 104 is different depending on the type of the unrecognized part, the display aspect of the importance mark 104 according to the importance 98 may be maintained as in the above-described embodiment. Further, the importance 98 may be changed depending on the type of the unrecognized part, and the first importance mark 104A, the second importance mark 104B, and the third importance mark 104C may be selectively displayed according to the changed importance 98.

In the above-described embodiment, an example of the form in which the importance 98 is defined at any one of three levels of “high”, “medium”, and “low” levels has been described. However, this is only an example, and the importance 98 may be at one or two of the “high”, “medium”, and “low” levels. In this case, the importance mark 104 may also be determined to be distinguishable for each level of the importance 98. For example, in a case where the importance 98 is only at the “high” and “medium” levels, the first importance mark 104A and the second importance mark 104B may be selectively displayed in the medical support image 41 according to the importance 98, and the third importance mark 104C may not be displayed in the medical support image 41.

In addition, the importance 98 may be divided into four or more levels. In this case, the importance mark 104 may also be determined to be distinguishable for each level of the importance 98.

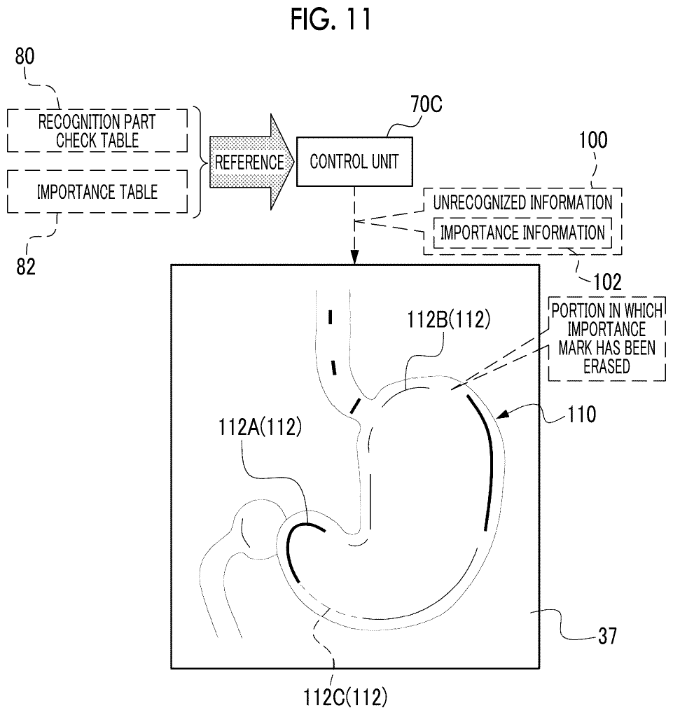

In the above-described embodiment, an example of the form in which the medical support image 41 is displayed on the screen 37 has been described. However, the technology of the present disclosure is not limited thereto. For example, as illustrated in FIG. 11, instead of the medical support image 41, a medical support image 110 may be displayed on the screen 37.

The unrecognized information 100 is displayed as the medical support image 110 on the screen 37 by the control unit 70C. The medical support image 110 is an example of the “schematic view” and a “second schematic view” according to the technology of the present disclosure. The importance information 102 is displayed as an importance mark 112 in the medical support image 110 by the control unit 70C instead of the importance mark 104 described in the above-described embodiment. The medical support image 110 is a schematic view perspectively showing a schematic aspect of the stomach. The importance mark 112 is a curved mark and is attached to each of the plurality of parts described in the above-described embodiment. In the example illustrated in FIG. 11, the importance mark 112 is attached to a portion along an inner wall of the stomach indicated by the medical support image 110.

In the example illustrated in FIG. 11, a first importance mark 112A is illustrated instead of the first importance mark 104A described in the above-described embodiment. In addition, in the example illustrated in FIG. 11, a second importance mark 112B is illustrated instead of the second importance mark 104B described in the above-described embodiment. Further, in the example illustrated in FIG. 11, a third importance mark 112C is illustrated instead of the third importance mark 104C described in the above-described embodiment.

The second importance mark 112B is displayed in a state in which it is emphasized more than the third importance mark 112C. In addition, the first importance mark 112A is displayed in a state in which it is emphasized more than the second importance mark 112B. In the example illustrated in FIG. 11, the thickness of the line of the second importance mark 112B is larger than the thickness of the line of the third importance mark 112C, and the thickness of the line of the first importance mark 112A is larger than the thickness of the line of the second importance mark 112B.

In the above-described embodiment, an example of the form in which the circular mark 108 corresponding to the part recognized by the recognition unit 70B is filled with a specific color has been described. However, in the example illustrated in FIG. 11, the importance mark 112 corresponding to the part recognized by the recognition unit 70B is erased. As a result, the portion to which the importance mark 104 is attached in the medical support image 110 is displayed in a state in which it is emphasized more than the portion in which the importance mark 112 has been erased in the medical support image 110.

This enables the doctor 14 to easily visually ascertain that the portion in which the importance mark 112 remains in the medical support image 110 is a portion corresponding to the part that has not been recognized by the recognition unit 70B and that the portion in which the importance mark 112 has been erased is a portion corresponding to the part that has been recognized by the recognition unit 70B. In addition, the importance mark 112 in the medical support image 110 is an example of the “first image” according to the technology of the present disclosure, and the portion in which the importance mark 112 has been erased in the medical support image 110 is an example of the “second image” according to the technology of the present disclosure.

In addition, in the example illustrated in FIG. 11, the doctor 14 visually ascertains which part of the stomach has not been recognized by the recognition unit 70B from the position of the importance mark 104 in the medical support image 110. In addition, the doctor 14 can recognize whether the first importance mark 112A, the second importance mark 112B, or the third importance mark 112C remains in the medical support image 110 to visually ascertain whether or not the part is likely to be missed in recognition by the recognition unit 70B. As described above, even in a case where the medical support image 110 is displayed on the screen 37, the same effects as those in the above-described embodiment can be expected.

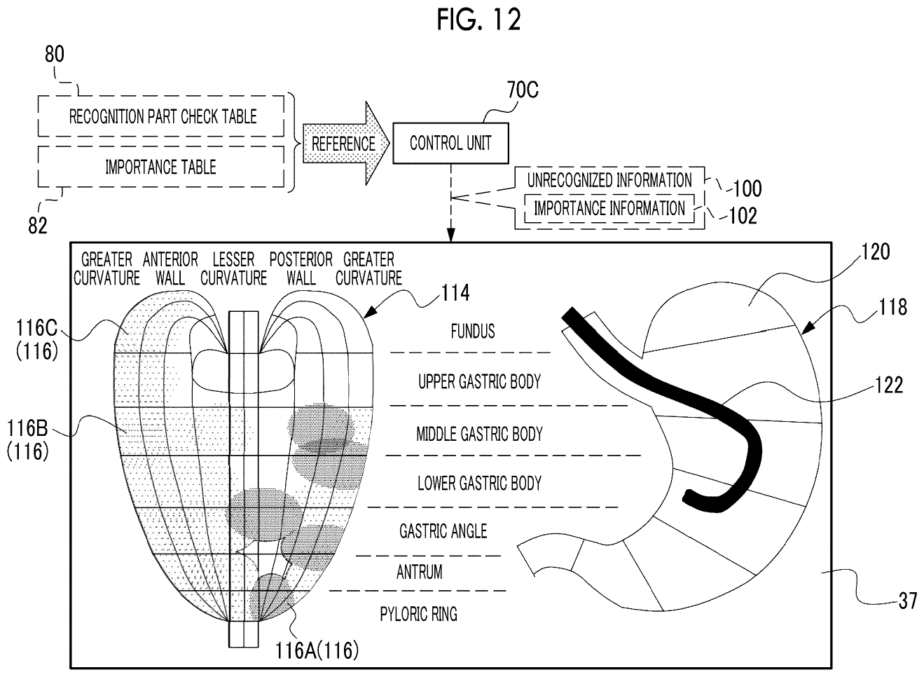

In addition, as illustrated in FIG. 12 as an example, instead of the medical support image 41 described in the above-described embodiment, a medical support image 114 may be displayed on the screen 37. In this case, the unrecognized information 100 is displayed as the medical support image 114 on the screen 37 by the control unit 70C. The medical support image 114 is an example of the “schematic view” and a “third schematic view” according to the technology of the present disclosure. The importance information 102 is displayed as an importance mark 116 in the medical support image 114 by the control unit 70C instead of the importance mark 104 described in the above-described embodiment. The medical support image 114 is a schematic view showing an aspect in which the stomach is schematically developed. In the medical support image 114, a plurality of parts are classified into each major category and each minor category. The importance mark 116 is an elliptical mark and is distributed in portions corresponding to the plurality of parts described in the above-described embodiment in the medical support image 114.

In the example illustrated in FIG. 12, a first importance mark 116A is illustrated instead of the first importance mark 104A described in the above-described embodiment. In addition, in the example illustrated in FIG. 12, a second importance mark 116B is illustrated instead of the second importance mark 104B described in the above-described embodiment. Further, in the example illustrated in FIG. 12, a third importance mark 116C is illustrated instead of the third importance mark 104C described in the above-described embodiment.

The second importance mark 116B is displayed in a state in which it is emphasized more than the third importance mark 116C. In addition, the first importance mark 116A is displayed in a state in which it is emphasized more than the second importance mark 116B. The first importance mark 116A, the second importance mark 116B, and the third importance mark 116C have different colors. The color of the second importance mark 116B is darker than the color of the third importance mark 116C, and the color of the first importance mark 116A is darker than the color of the second importance mark 116B.

In the above-described embodiment, an example of the form in which the circular mark 108 corresponding to the part recognized by the recognition unit 70B is filled with a specific color has been described. However, in the example illustrated in FIG. 12, the importance mark 116 corresponding to the part recognized by the recognition unit 70B is erased. As a result, a portion to which the importance mark 116 is applied in the medical support image 114 is displayed in a state in which it is emphasized more than a portion in which the importance mark 116 has been erased in the medical support image 114.

As a result, the doctor 14 can easily visually ascertain that the portion in which the importance mark 116 remains in the medical support image 114 corresponds to the part that has not been recognized by the recognition unit 70B and that the portion in which the importance mark 116 has been erased corresponds to the part that has been recognized by the recognition unit 70B. In addition, the importance mark 116 in the medical support image 114 is an example of the “first image” according to the technology of the present disclosure, and the portion in which the importance mark 116 has been erased in the medical support image 114 is an example of the “second image” according to the technology of the present disclosure.

Further, in the example illustrated in FIG. 12, the doctor 14 visually ascertains which part of the stomach has not been recognized by the recognition unit 70B from the position of the importance mark 116 in the medical support image 114. In addition, the doctor 14 can recognize whether the first importance mark 116A, the second importance mark 116B, or the third importance mark 116C remains in the medical support image 114 to visually ascertain whether or not the part is likely to be missed in recognition by the recognition unit 70B. As described above, even in a case where the medical support image 114 is displayed on the screen 37, the same effects as those in the above-described embodiment can be expected.

In addition, in the example illustrated in FIG. 12, the control unit 70C displays a reference image 118 on the screen 37 in a state in which the reference image 118 is disposed side by side with the medical support image 114. The reference image 118 is divided into a plurality of regions 120. In the example illustrated in FIG. 12, the fundus, the upper gastric body, the middle gastric body, the lower gastric body, the gastric angle, the antrum, and the pyloric ring are illustrated as an example of the plurality of regions 120. The plurality of regions 120 are displayed on the screen 37 to be comparable with portions of the parts classified into the major categories in the medical support image 114. In addition, an insertion portion image 122 capable of specifying the current position of the insertion portion 44 of the endoscope main body 18 is displayed in the reference image 118. The insertion portion image 122 is an image that imitates the insertion portion 44. The shape and position of the insertion portion image 122 are linked to the actual shape and position of the insertion portion 44.

The actual shape and position of the insertion portion 44 are specified by executing the AI-type image recognition process. For example, the control unit 70C specifies the actual shape and position of the insertion portion 44 by performing the process using the trained model on the content of the operation of the insertion portion 44 and the endoscopic images 40 of one or more frames, generates the insertion portion image 122 on the basis of the specification results, and displays the insertion portion image 122 to be superimposed on the reference image 118 on the screen 37.

Here, for example, the trained model used by the control unit 70C is obtained by performing machine learning on the neural network using training data in which the content of the operation of the insertion portion 44, images corresponding to the endoscopic images 40 of one or more frames, and the like are example data and the shape and position of the insertion portion 44 are correct answer data.

In the example illustrated in FIG. 8, an example of the form in which the medical support image 41 is displayed on the screen 37 has been described. In the example illustrated in FIG. 11, an example of the form in which the medical support image 110 is displayed on the screen 37 has been described. In the example illustrated in FIG. 12, an example of the form in which the medical support image 114 is displayed on the screen 37 has been described. However, these are only examples. For example, the medical support images 41, 110, and 114 may be selectively displayed, or two or more of the medical support images 41, 110, and 114 may be displayed in a state in which the medical support images are arranged side by side (that is, in a state in which the medical support images are comparable).

In the above-described embodiment, an example of the form in which the importance 98 assigned to a plurality of parts is determined according to the data of the past examination performed on the plurality of parts has been described. However, the technology of the present disclosure is not limited thereto. For example, the importance 98 assigned to the plurality of parts may be determined according to the position of the unrecognized part in the stomach. The omission of the recognition of a part that is spatially farthest from the position of the distal end part 46 by the recognition unit 70B is more likely to occur than the omission of the recognition of a part that is spatially closer to the position of the distal end part 46. Therefore, an example of the position of the unrecognized part in the stomach is the position of the unrecognized part that is spatially farthest from the position of the distal end part 46. In this case, the position of the unrecognized part that is spatially farthest from the position of the distal end part 46 changes depending on the position of the distal end part 46. Therefore, the importance 98 assigned to a plurality of parts changes depending on the position of the distal end part 46 and the position of the unrecognized part in the stomach. As described above, since the importance 98 assigned to the plurality of parts is determined according to the position of the unrecognized part in the stomach, it is possible to suppress the omission of the recognition of the part with the high importance 98 determined according to the position of the unrecognized part in the stomach by the recognition unit 70B.

In the above-described embodiment, an example of the form in which the importance 98 assigned to the plurality of parts is determined in response to an instruction given from the outside has been described. However, the technology of the present disclosure is not limited thereto. For example, the importance 98 corresponding to a part that is scheduled to be recognized by the recognition unit 70B before a designated part (for example, a part corresponding to a predetermined checkpoint), among a plurality of parts, may be set to be higher than the importance 98 corresponding to a part that is scheduled to be recognized after the designated part, among the plurality of parts. This makes it possible to suppress the omission of the recognition of the part that is scheduled to be recognized by the recognition unit 70B before the designated part.

In the above-described embodiment, an example of the form in which the unrecognized part is set regardless of the part classified into the major category and the part classified into the minor category, among a plurality of parts, has been described. However, the technology of the present disclosure is not limited thereto. For example, the omission of the recognition of the part classified into the minor category by the recognition unit 70B is more likely to occur than the omission of the recognition of the part classified into the major category by the recognition unit 70B. Therefore, the unrecognized part may be set only for the part classified into the minor category among the plurality of parts. In this case, the omission of the recognition by the recognition unit 70B can be less likely to occur as compared to a case in which the omission of the recognition of both the part classified into the major category and the part classified into the minor category by the recognition unit 70B is suppressed.

In the above-described embodiment, an example of the form has been described in which, in a case in which the part classified into the minor category has not been recognized by the recognition unit 70B, the unrecognized information 100 is output on condition that the recognition unit 70B recognizes the part classified into the major category which is scheduled to be recognized by the recognition unit 70B after the part that has not been recognized by the recognition unit 70B. However, the technology of the present disclosure is not limited thereto.

For example, in a case in which the part classified into the minor category has not been recognized by the recognition unit 70B, the unrecognized information 100 may be output on condition that the recognition unit 70B recognizes a part classified into the minor category which is scheduled to be recognized by the recognition unit 70B after the part (that is, the part classified into the minor category) that has not been recognized by the recognition unit 70B. In this case, in a situation in which there is a high probability that the omission of recognition will occur for a part (here, for example, a part classified into the minor category) in the observation target 21, the doctor 14 can understand that the omission of recognition has occurred for the part in the observation target 21.

Here, a plurality of parts classified into the minor categories among the plurality of parts are an example of a “plurality of minor category parts” according to the technology of the present disclosure. A part that has not been recognized by the recognition unit 70B among the plurality of parts classified into the minor categories is an example of a “first minor category part” according to the technology of the present disclosure. The part classified into the minor category that is scheduled to be recognized by the recognition unit 70B after the part (that is, the part classified into the minor category) that has not been recognized by the recognition unit 70B is an example of a “second minor category part” according to the technology of the present disclosure.

In addition, for example, in a case where the part classified into the minor category has not been recognized by the recognition unit 70B, the unrecognized information 100 may be output on condition that the recognition unit 70B recognizes the plurality of parts classified into the minor categories which are scheduled to be recognized by the recognition unit 70B after the part (that is, the part classified into the minor category) that has not been recognized by the recognition unit 70B. In this case, in a situation in which there is a high probability that the omission of the recognition of a part (here, for example, a part classified into the minor category) in the observation target 21 will occur, the doctor 14 can understand that the omission of the recognition of the part in the observation target 21 has occurred.

Here, the plurality of parts classified into the minor categories which are scheduled to be recognized by the recognition unit 70B after the part (that is, the part classified into the minor category) that has not been recognized by the recognition unit 70B are an example of a “plurality of second minor category parts” according to the technology of the present disclosure.

In the above-described embodiment, an example of the form in which the unrecognized information 100 is output from the control unit 70C to the display device 13 has been described. However, the technology of the present disclosure is not limited thereto. For example, the unrecognized information 100 may be stored in headers or the like of various images such as the endoscopic images 40. For example, in a case where the part that has not been recognized by the recognition unit 70B is classified into the minor category, the fact that the part is classified into the minor category and/or information that can specify the part may be stored in the headers or the like of various images such as the endoscopic images 40. In addition, for example, in a case where the part that has not been recognized by the recognition unit 70B is classified into the major category, the fact that the part is classified into the major category and/or information that can specify the part may be stored in the headers or the like of various images such as the endoscopic images 40.

Further, a recognition order (that is, the order of the parts recognized by the recognition unit 70B) including the major category and the minor category and/or information related to a final unrecognized part (that is, the part that has not been recognized by the recognition unit 70B) may be transmitted to an examination system that is connected to the endoscope 12 such that it can communicate therewith and may be stored as examination data by the examination system or may be posted in an examination diagnosis report.

In the above-described embodiment, an example of the form has been described in which the camera 48 sequentially images a plurality of parts on the greater-curvature-side route 106A from the upstream side (that is, an entrance side of the stomach) to the downstream side of the stomach (that is, an exit side of the stomach) and sequentially images the lesser-curvature-side route 106B from the upstream side to the downstream side of the stomach (that is, the parts are imaged along the scheduled recognition order 97). However, the technology of the present disclosure is not limited thereto. For example, in a case where the recognition unit 70B sequentially recognizes a first part (for example, the posterior wall of the upper gastric body) on the upstream side in an insertion direction of the insertion portion 44 inserted into the stomach and a second part (for example, the posterior wall of the lower gastric body) on the downstream side, the processor 70 estimates that imaging is performed along a first route (here, for example, the greater-curvature-side route 106A) determined from the upstream side to the downstream side of the insertion portion 44, and the unrecognized information 100 is output along the first route. In addition, for example, in a case where the recognition unit 70B sequentially recognizes a third part (for example, the posterior wall of the lower gastric body) on the downstream side in the insertion direction of the insertion portion 44 inserted into the stomach and a fourth part (for example, the posterior wall of the upper gastric body) on the upstream side, the processor 70 estimates that imaging is performed along a second route (here, for example, the lesser-curvature-side route 106B) determined from the downstream side to the upstream side of the insertion portion 44, and the unrecognized information 100 is output along the second route. Therefore, it is possible to easily specify whether the part on the greater-curvature-side route 106A is not recognized by the recognition unit 70B or the part on the lesser-curvature-side route 106B is not recognized by the recognition unit 70B.

In addition, here, the greater-curvature-side route 106A is given as an example of the first route, and the lesser-curvature-side route 106B is given as an example of the second route. However, the first route may be the lesser-curvature-side route 106B, and the second route may be the greater-curvature-side route 106A. Further, here, the upstream side in the insertion direction indicates the entrance side of the stomach (that is, an esophageal side), and the downstream side in the insertion direction indicates the exit side of the stomach (that is, a duodenal side).

In the above-described embodiment, an example of the form in which the medical support process is performed by the processor 70 of the computer 64 included in the endoscope 12 has been described. However, the technology of the present disclosure is not limited thereto. The device that performs the medical support process may be provided outside the endoscope 12. An example of the device provided outside the endoscope 12 is at least one server and/or at least one personal computer that is connected to the endoscope 12 such that it can communicate therewith. In addition, the medical support process may be dispersively performed by a plurality of devices.

Further, in the above-described embodiment, an example of the form in which the medical support processing program 76 is stored in the NVM 74 has been described. However, the technology of the present disclosure is not limited thereto. For example, the medical support processing program 76 may be stored in a portable non-transitory storage medium such as an SSD or a USB memory. The medical support processing program 76 stored in the non-transitory storage medium is installed in the computer 64 of the endoscope 12. The processor 70 performs the medical support process according to the medical support processing program 76.

In addition, the medical support processing program 76 may be stored in a storage device of another computer or a server that is connected to the endoscope 12 through a network. Then, the medical support processing program 76 may be downloaded and installed in the computer 64 in response to a request from the endoscope 12.

In addition, all of the medical support processing program 76 does not need to be stored in the storage device of another computer or a server connected to the endoscope 12 or the NVM 74, and a portion of the medical support processing program 76 may be stored therein.

The following various processors can be used as hardware resources for performing the medical support process. An example of the processor is a CPU which is a general-purpose processor that executes software, that is, a program, to function as the hardware resource performing the medical support process. In addition, an example of the processor is a dedicated electronic circuit which is a processor having a dedicated circuit configuration designed to perform a specific process, such as an FPGA, a PLD, or an ASIC. Any processor has a memory provided therein or connected thereto. Any processor uses the memory to perform the medical support process.

The hardware resource for performing the medical support process may be configured by one of the various processors or by a combination of two or more processors of the same type or different types (for example, a combination of a plurality of FPGAs or a combination of a CPU and an FPGA). Further, the hardware resource for performing the medical support process may be one processor.

A first example of the configuration in which the hardware resource is configured by one processor is an aspect in which one processor is configured by a combination of one or more CPUs and software and functions as the hardware resource for performing the medical support process. A second example of the configuration is an aspect in which a processor that implements the functions of the entire system including a plurality of hardware resources for performing the medical support process using one IC chip is used. A representative example of this aspect is an SoC. As described above, the medical support process is achieved using one or more of the various processors as the hardware resources.

In addition, specifically, an electronic circuit obtained by combining circuit elements, such as semiconductor elements, can be used as the hardware structure of the various processors. Further, the above-described medical support process is only an example. Therefore, it goes without saying that unnecessary steps may be deleted, new steps may be added, or the processing order may be changed, without departing from the gist.

The content described and illustrated above is a detailed description of portions related to the technology of the present disclosure and is only an example of the technology of the present disclosure. For example, the description of the configurations, functions, operations, and effects is the description of examples of the configurations, functions, operations, and effects of the portions related to the technology of the present disclosure. Therefore, it goes without saying that unnecessary portions may be deleted or new elements may be added or replaced in the content described and illustrated above, without departing from the gist of the technology of the present disclosure. In addition, the description of, for example, common technical knowledge that does not need to be particularly described to enable the implementation of the technology of the present disclosure is omitted in the content described and illustrated above in order to avoid confusion and to facilitate the understanding of the portions related to the technology of the present disclosure.

In the specification, “A and/or B” is synonymous with “at least one of A or B”. That is, “A and/or B” means only A, only B, or a combination of A and B. Further, in the specification, the same concept as “A and/or B” is applied to a case where the connection of three or more matters is expressed by “and/or”.

All of the documents, the patent applications, and the technical standards described in the specification are incorporated by reference herein to the same extent as each individual document, each patent application, and each technical standard is specifically and individually stated to be incorporated by reference.

What is claimed is:

1. A medical support device comprising:
a processor,
wherein the processor is configured to:
recognize a plurality of parts in an observation target on the basis of a plurality of medical images including the observation target; and
in a case where an unrecognized part in the observation target is present in the plurality of parts, output unrecognized information capable of specifying that the unrecognized part is present, and wherein:
the plurality of parts include a subsequent part that is scheduled to be recognized by the processor after the unrecognized part, and
the processor is configured to output the unrecognized information on condition that the subsequent part is recognized.

2. The medical support device according to claim 1,
wherein the processor is configured to output the unrecognized information on the basis of a first order, which is an order in which the plurality of parts are recognized by the processor, and a second order, which is an order in which a plurality of scheduled parts that include the unrecognized part and that are scheduled to be recognized by the processor are recognized by the processor.

3. The medical support device comprising:
a processor,
wherein the processor is configured to:
recognize a plurality of parts in an observation target on the basis of a plurality of medical images including the observation target; and
in a case where an unrecognized part in the observation target is present in the plurality of parts, output unrecognized information capable of specifying that the unrecognized part is present, and wherein:
importance is assigned to the plurality of parts, and
the unrecognized information includes importance information capable of specifying the importance.

4. The medical support device according to claim 3,
wherein the importance corresponding to a part which is scheduled to be recognized by the processor before a designated part, among the plurality of parts, is higher than the importance corresponding to a part which is scheduled to be recognized after the designated part, among the plurality of parts.

5. The medical support device according to claim 3,
wherein the plurality of parts are classified into a major category and a minor category included in the major category, and
the importance corresponding to a part which is classified into the minor category, among the plurality of parts, is higher than the importance corresponding to a part which is classified into the major category, among the plurality of parts.

6. The medical support device according to claim 1,
wherein the plurality of parts are classified into a major category and a minor category included in the major category, and
the unrecognized part is a part which is classified into the minor category, among the plurality of parts.

7. The medical support device according to claim 3,
wherein the plurality of parts are classified into a major category and a minor category included in the major category,
the unrecognized part is a part which is classified into the minor category, among the plurality of parts,
the major category is roughly classified into a first major category and a second major category,
the part classified into the second major category is scheduled to be recognized by the processor after the part classified into the first major category,
the unrecognized part is a part which belongs to the minor category included in the first major category, among the plurality of parts, and
the processor is configured to output the unrecognized information on condition that a part which is classified into the second major category, among the plurality of parts, is recognized.

8. The medical support device according to claim 3,
wherein the plurality of parts are classified into a major category and a minor category included in the major category,
the unrecognized part is a part which is classified into the minor category, among the plurality of parts,
the plurality of parts include a plurality of minor category parts classified into the minor category,
the plurality of minor category parts include a first minor category part and a second minor category part that is scheduled to be recognized by the processor after the first minor category part,
the unrecognized part is the first minor category part, and
the processor is configured to output the unrecognized information on condition that the second minor category part is recognized.

9. The medical support device according to claim 3,
wherein the plurality of parts are classified into a major category and a minor category included in the major category,
the unrecognized part is a part which is classified into the minor category, among the plurality of parts,
the plurality of parts include a plurality of minor category parts belonging to the minor category,
the plurality of minor category parts include a first minor category part and a plurality of second minor category parts that are scheduled to be recognized by the processor after the first minor category part,
the unrecognized part is the first minor category part, and
the processor is configured to output the unrecognized information on condition that the plurality of second minor category parts are recognized.

10. The medical support device according to claim 1,
wherein an output destination of the unrecognized information includes a display device.

11. The medical support device according to claim 10,
wherein the unrecognized information includes a first image capable of specifying the unrecognized part and a second image capable of specifying parts other than the unrecognized part, among the plurality of parts, and
the first image and the second image are displayed on the display device in an aspect in which the first image and the second image are distinguishable.

12. The medical support device according to claim 11,
wherein a schematic view in which the observation target is divided into a plurality of regions corresponding to the plurality of parts is displayed on the display device, and the first image and the second image are displayed in the schematic view in an aspect in which the first image and the second image are distinguishable.

13. The medical support device according to claim 12,
wherein the observation target is a luminal organ, and
the schematic view is a first schematic view showing a schematic aspect of at least one route for observing the luminal organ, a second schematic view perspectively showing a schematic aspect of the luminal organ, and/or a third schematic view showing an aspect in which the luminal organ is schematically developed.

14. The medical support device according to claim 11,
wherein the first image is displayed on the display device in a state in which the first image is emphasized more than the second image.

15. The medical support device according to claim 11,
wherein importance is assigned to the plurality of parts, and
a display aspect of the first image differs depending on the importance.

16. The medical support device according to claim 11,
wherein a display aspect of the first image differs depending on a type of the unrecognized part.

17. The medical support device according to claim 1,
wherein the medical image is an image obtained from an endoscope inserted into a body, and
the processor is configured to:
in a case in which a first part on an upstream side in an insertion direction of the endoscope inserted into the body and a second part on a downstream side are sequentially recognized, output the unrecognized information along a first route determined from the upstream side to the downstream side in the insertion direction; and
in a case in which a third part on the downstream side in the insertion direction and a fourth part on the upstream side are sequentially recognized, output the unrecognized information along a second route determined from the downstream side to the upstream side in the insertion direction.

18. An endoscope comprising:
the medical support device according to claim 1; and
an image acquisition device that acquires an endoscopic image as the medical image.

19. A medical support method comprising:
recognizing a plurality of parts in an observation target on the basis of a plurality of medical images including the observation target; and
in a case where an unrecognized part in the observation target is present in the plurality of parts, outputting unrecognized information capable of specifying that the unrecognized part is present, and wherein:
the plurality of parts include a subsequent part that is scheduled to be recognized by the processor after the unrecognized part, and
the medical support method comprises outputting the unrecognized information on condition that the subsequent part is recognized.

20. A non-transitory computer-readable storage medium storing a program executable by a computer to execute a process comprising:
recognizing a plurality of parts in an observation target on the basis of a plurality of medical images including the observation target; and
in a case where an unrecognized part in the observation target is present in the plurality of parts, outputting unrecognized information capable of specifying that the unrecognized part is present, and wherein:
the plurality of parts include a subsequent part that is scheduled to be recognized by the processor after the unrecognized part, and
the process comprises outputting the unrecognized information on condition that the subsequent part is recognized.
